(19) United States
(12) Patent Application Publication
(10) Pub. No.: US 2020/0341011 A1
(43) Pub. Date: Oct. 29, 2020

(54) ASTROCYTE EXOSOME COMPLEMENT-BASED ASSAY FOR NEUROINFLAMMATION IN ALZHEIMER'S DISEASE AND USES THEREOF

(71) Applicant: Edward J. Goetzl, San Francisco, CA (US)

(72) Inventor: Edward J. Goetzl, San Francisco, CA (US)

(21) Appl. No.: 16/764,858

(22) PCT Filed: Nov. 16, 2018
(86) PCT No.: PCT/US2018/061690

§ 371 Date: May 16, 2020

Related U.S. Application Data
(60) Provisional application No. 62/588,228, filed on Nov. 17, 2017.

(51) Int. Cl. G01N 33/68

(52) U.S. Cl. CPC G01N 33/6896; G01N 2800/2821; G01N 2333/4716; A61K 45/06; G01N 2333/545; G01N 2333/5412; G01N 2333/525; G01N 2333/70596

(57) ABSTRACT

The present invention relates to astrocyte-derived exosomal complement protein biomarkers and diagnostic and prognostic methods for neurological disease (e.g., Alzheimer's disease). The invention also provides compositions for detecting astrocyte-derived exosomal complement protein biomarkers as well as compositions and methods useful for treating neurological disease (e.g., Alzheimer's disease).

### RELATED APPLICATIONS

This application claims priority to the U.S. Provisional Patent Application Ser. No. 62/588,228, filed on Nov. 17, 2017, which is hereby incorporated by reference herein in its entirety.

### FIELD OF THE INVENTION

The present invention relates to astrocyte-derived exosomal complement protein biomarkers and diagnostic and prognostic methods for neurological disease (e.g., Alzheimer's disease). The invention also provides compositions for detecting astrocyte-derived exosomal complement protein biomarkers as well as compositions and methods useful for treating neurological disease (e.g., Alzheimer's disease).

### BACKGROUND OF THE INVENTION

Astrocytes are abundant glial cells in the human central nervous system (CNS), that normally have a major neuronal trophic role through diverse homeostatic maintenance activities. Neuronal supportive functions of astrocytes include promotion of neuronal development, nutrition, survival, dendrite outgrowth and synapse formation (Sofroniew and Vinters. Acta Neuropathol 2010; 119, 7-35; J. L. Zamanian et al. J Neurosci 32, 2012; 6391-6410; M. A. Anderson et al., Nature 2016; 532, 195-200). Most inflammatory, ischemic and neurodegenerative diseases of the CNS elicit a highly coordinated multicellular response that encompasses an increase in the total number of astrocytes and differentiation into reactive astrocytes of types A1 and/or A2 (Liddelow et al. Immunity 2017; 46, 957-967; Haim et al. Front Cell Neurosci 2015; 9, 278; Goetzl et al. Faseb J 2017; 31, 1792-1795). Activated microglia are critical inducers of A1 inflammatory-neurotoxic astrocytes through NFκB-dependent pathways, but a greater understanding of mechanisms and specific mediators is required (Liddelow et al., Nature 2017; 541, 481-487). A2-type reactive astrocytes upregulate expression of neuronal protective functions and factors. In contrast, A1-type reactive astrocytes lose neuronal trophic potential and instead increase expression of pro-inflammatory pathways as well as toxic activities that damage synapses and destroy neurons. It is currently unclear which of the neuronal toxic mediators generated and secreted by A1-type astrocytes are pathogenically critical in human neurodegenerative diseases.

Findings in postmortem brain tissues of patients with several neurodegenerative and neuroinflammatory diseases have begun to delineate specific components of A1-type astrocyte-mediated neuronal toxicity. For example, approximately 60% of A1 (glial fibrillary acidic protein [GFAP]-positive) astrocytes in the prefrontal cortex of patients with Alzheimer's disease (AD) express an abnormally high level of complement component 3 (C3), that is characteristically upregulated in induced A1-type astrocytes and has potential neuronal cytotoxic activity. The absence of C3 from A2 (S100A10-positive) astrocytes in the same regions of brain tissues of patients with AD confirms the likely absence of complement-mediated neuronal cytotoxic activity of A2 astrocytes.

Enriched populations of astrocyte-derived exosomes (ADEs) obtained from human plasma by sequential precipitation and immunochemical absorption contain much higher levels of the astrocyte biomarkers glutamine synthetase and GFAP than plasma neuron-derived exosomes (NDEs). In contrast, NDEs have much higher levels than ADEs of the neuronal markers neurofilament light chain and neuron-specific enolase (Goetzl et al., Faseb J 2016; 30, 3853-3859). Levels of β-site amyloid precursor protein-cleaving enzyme 1 (BACE-1) and soluble amyloid precursor protein β (sAPPβ) of the Aβ42 peptide-generating system are higher in ADEs than NDEs of cognitively normal subjects and are higher in ADEs of patients with AD than in those of matched controls (Goetzl et al., Faseb J 2016; 30, 3853-3859). Dysfunction in complement pathways may be an etiological factor in the neuroinflammation of Alzheimer's disease and other neurological diseases.

Thus, there is a need in the art for biomarkers and methods for detecting astrocyte-derived exosomal complement biomarkers associated with pathogenesis of neurological diseases, such as, for example Alzheimer's disease. Additionally, there is a need in the art for compositions for detecting biomarkers as well as compositions and methods useful for treating Alzheimer's disease and other neurological diseases. The present invention meets this need by providing accurate, noninvasive methods for detecting complement biomarkers that are diagnostic for neurological diseases. The present invention further provides novel methods, assays, kits, and compositions for diagnosing, prognosing, predicting, and treating Alzheimer's disease and other neurological diseases.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention is based on the discovery of biomarkers from astrocyte-derived exosomes that can be used to detect complement system abnormalities associated with pathogenesis of neurological diseases, including Alzheimer's disease. These biomarkers can be used alone or in combination with one or more additional biomarkers or relevant clinical parameters in prognosis, diagnosis, or monitoring treatment of complement system abnormalities associated with neurological diseases.

Biomarkers that can be used in the practice of the invention include, but are not limited to, inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1).

In some embodiments, the present invention provides a method comprising: a) providing a biological sample comprising astrocyte-derived exosomes from a subject; and b) detecting the presence of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) in the sample.

In other embodiments, the present invention provides a method comprising: a) providing a biological sample comprising astrocyte-derived exosomes from a subject; b) enriching the sample for astrocyte-derived exosomes; and c) detecting the presence of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) in the sample. In some embodiments, the methods of the present invention further comprise determining the level or concentration of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) in the sample. In some embodiments, the subject has a neurological disease. In other embodiments, the neurological disease is selected from the group consisting of: Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In some embodiments, the present invention provides a method of detecting markers in a biological sample, the method comprising: a) providing a biological sample comprising astrocyte-derived exosomes from a subject; b) isolating astrocyte-derived exosomes from the biological sample; and c) detecting the presence of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) in the exosomes. In some embodiments, the one or more biomarkers comprises inflammatory cytokines, interleukin 6 (IL-6), tumor necrosis factor alpha (TNF-α), and interleukin 1 beta (IL-1β). In some embodiments, the one or more biomarkers comprises complement proteins, C1q, C4b, C5b, C3d, factor B, factor D, Fragment Bb, C3b, and C5b-C9 terminal complement complex (TCC). In some embodiments, the one or more biomarkers comprises complement regulatory proteins, membrane inhibitor of reactive lysis (CD59), membrane cofactor protein (CD46), decay-accelerating factor (DAF) and complement receptor type 1 (CR1). In some embodiments, the subject has a neurological disease. In other embodiments, the neurological disease is selected from the group consisting of: Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In other embodiments, the present invention provides a method of detecting markers in a biological sample, the method comprising: a) providing; i) a biological sample comprising astrocyte-derived exosomes from a subject and ii) immunoassay reagents for the detection of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1); and b) detecting the presence of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) in the sample using said reagents. In some embodiments, the one or more biomarkers comprises inflammatory cytokines, interleukin 6 (IL-6), tumor necrosis factor alpha (TNF-α), and interleukin 1 beta (IL-1β). In some embodiments, the one or more biomarkers comprises complement proteins, C1q, C4b, C5b, C3d, factor B, factor D, Fragment Bb, C3b, and C5b-C9 terminal complement complex (TCC). In some embodiments, the one or more biomarkers comprises complement regulatory proteins, membrane inhibitor of reactive lysis (CD59), membrane cofactor protein (CD46), decay-accelerating factor (DAF) and complement receptor type 1 (CR1).

In other embodiments, the present invention provides a method of detecting markers in a biological sample, the method comprising: a) providing; i) a biological sample comprising astrocyte-derived exosomes from a subject and ii) immunoassay reagents for detection of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1); b) isolating astrocyte-derived exosomes from the biological sample and c) detecting the presence of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) in the exosomes using said reagents. In some embodiments, the one or more biomarkers comprises inflammatory cytokines, interleukin 6 (IL-6), tumor necrosis factor alpha (TNF-α), and interleukin 1 beta (IL-1β). In some embodiments, the one or more biomarkers comprises complement proteins, C1q, C4b, C5b, C3d, factor B, factor D, Fragment Bb, C3b, and C5b-C9 terminal complement complex (TCC). In some embodiments, the one or more biomarkers comprises complement regulatory proteins, membrane inhibitor of reactive lysis (CD59), membrane cofactor protein (CD46), decay-accelerating factor (DAF) and complement receptor type 1 (CR1). In some embodiments, the subject has a neurological disease. In other embodiments, the neurological disease is selected from the group consisting of: Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In some embodiments, the reagents comprise antibodies for performing an immunoassay. In some embodiments, the immunoassay is selected from the group consisting of an ELISA, radio-immunoassay, automated immunoassay, cytometric bead assay, and immunoprecipitation assay. In other embodiments, the biological sample can be any bodily fluid comprising astrocyte-derived exosomes, including, but not limited to, whole blood, plasma, serum, lymph, amniotic fluid, urine, saliva, and umbilical cord blood. In some embodiments, the marker is a full-size marker. In other embodiments said marker is a fragment of the full-size marker. In other embodiments, the detecting the presence of the marker in the biological sample comprises detecting the amount of the marker in the biological sample. In some embodiments, the method further comprises the step of determining a treatment course of action based on the detection of the marker or the diagnosis of a neurological disease.

In some embodiments, the subject has been diagnosed with a neurological disease or suspected of having a neurological disease, which may include, but not limited to, Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease. In other embodiments, the subject is at-risk of developing a neurological disease, which may include, but not limited to, Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In some embodiments, isolating astrocyte-derived exosomes from the biological sample comprises: contacting the biological sample with an agent under conditions wherein an astrocyte-derived exosome present in the biological sample binds to the agent to form an astrocyte-derived exosome-agent complex; and isolating the astrocyte-derived exosome from the astrocyte-derived exosome-agent complex to obtain a sample containing the astrocyte-derived exosome, wherein the purity of the astrocyte-derived exosomes present in said sample is greater than the purity of the astrocyte-derived exosomes present in said biological sample. The agent may be an antibody that specifically binds to an astrocyte-derived exosome surface marker (e.g., Glutamine Aspartate Transporter (GLAST)). In some aspects of the present embodiment, the contacting comprises incubating or reacting. Example 1 describes isolation of astrocyte-derived exosomes from a biological sample, for example, by immunoabsorption using an anti-human Glutamine Aspartate Transporter (GLAST) (ACSA-1) biotinylated antibody specific for this surface protein.

Biomarker proteins can be measured, for example, by performing immunohistochemistry, immunocytochemistry, immunofluorescence, immunoprecipitation, Western blotting, or an enzyme-linked immunosorbent assay (ELISA). In certain embodiments, the level of a biomarker is measured with an immunoassay. For example, the level of the biomarker can be measured by contacting an antibody with the biomarker, wherein the antibody specifically binds to the biomarker, or a fragment thereof containing an antigenic determinant of the biomarker. Antibodies that can be used in the practice of the invention include, but are not limited to, monoclonal antibodies, polyclonal antibodies, chimeric antibodies, recombinant fragments of antibodies, Fab fragments, Fab′ fragments, F(ab′)₂ fragments, Fᵥ fragments, or scFᵥ fragments. In one embodiment, the method comprises measuring amounts of an in vitro complex comprising a labeled antibody bound to an astrocyte-derived exosome biomarker. In one aspect, the astrocyte-derived exosome biomarker is selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1). In some embodiments, increased levels of the biomarker inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC) compared to reference value ranges of the biomarkers for a control subject indicate that the subject has a neurological disease (e.g., Alzheimer's disease) or is at-risk of developing a neurological disease. In some aspects, the control subject is a subject without a neurological disease. In some embodiments, decreased levels of the biomarker complement regulatory proteins, membrane inhibitor of reactive lysis (CD59), membrane cofactor protein (CD46), decay-accelerating factor (DAF) and complement receptor type 1 (CR1) compared to reference value ranges of the biomarkers for a control subject indicate that the subject has a neurological disease (e.g., Alzheimer's disease) or is at-risk of developing a neurological disease. In some aspects, the control subject is a subject without a neurological disease. In some embodiments, the one or more biomarkers comprises inflammatory cytokines, interleukin 6 (IL-6), tumor necrosis factor alpha (TNF-α), and interleukin 1 beta (IL-1β). In some embodiments, the one or more biomarkers comprises complement proteins, C1q, C4b, C5b, C3d, factor B, factor D, Fragment Bb, C3b, and C5b-C9 terminal complement complex (TCC). In some embodiments, the one or more biomarkers comprises complement regulatory proteins, membrane inhibitor of reactive lysis (CD59), membrane cofactor protein (CD46), decay-accelerating factor (DAF) and complement receptor type 1 (CR1).

The levels of the biomarkers from astrocyte-derived exosomes from a subject can be compared to reference value ranges for the biomarkers found in one or more samples of astrocyte-derived exosomes from one or more subjects without a neurological disease (e.g., control sample, healthy subject without neurological disease). Alternatively, the levels of the biomarkers from astrocyte-derived exosomes from a subject can be compared to reference values ranges for the biomarkers found in one or more samples of astrocyte-derived exosomes from one or more subjects with a neurological disease. In some embodiments, the subject has a neurological disease. In other embodiments, the neurological disease is selected from the group consisting of: Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In some embodiments, the invention provides a method for monitoring the efficacy of a therapy for treating a neurological disease in a patient, the method comprising: a) providing a first biological sample comprising astrocyte-derived exosomes from the patient before the patient undergoes the therapy and a second biological sample comprising astrocyte-derived exosomes after the patient undergoes the therapy; b) isolating astrocyte-derived exosomes from the first biological sample and the second biological sample; and c) detecting one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, and C5b-C9 terminal complement complex (TCC) for the astrocyte-derived exosomes from the first biological sample and the second biological sample; and d) comparing the levels of the one or more biomarkers for the astrocyte-derived exosomes from the first biological sample to the levels of the one or more biomarkers for the astrocyte-derived exosomes from the second biological sample, wherein decreased levels of the one or more biomarkers for the astrocyte-derived exosomes from the second biological sample compared to the levels of the one or more biomarkers for the astrocyte-derived exosomes from the first biological sample indicate that the patient is improving, and increased levels of the one or more biomarkers for the astrocyte-derived exosomes from the second biological sample compared to the levels of the one or more biomarkers for the astrocyte-derived exosomes from the first biological sample indicate that the patient is worsening or not responding to the therapy. In some embodiments, the one or more biomarkers comprises inflammatory cytokines, interleukin 6 (IL-6), tumor necrosis factor alpha (TNF-α), and interleukin 1 beta (IL-1β). In some embodiments, the one or more biomarkers comprises complement proteins, C1q, C4b, C5b, C3d, factor B, factor D, Fragment Bb, C3b, and C5b-C9 terminal complement complex (TCC). In some embodiments, the neurological disease is selected from the group consisting of: Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In some embodiments, the invention provides a method for monitoring the efficacy of a therapy for treating a neurological disease in a patient, the method comprising: a) providing a first biological sample comprising astrocyte-derived exosomes from the patient before the patient undergoes the therapy and a second biological sample comprising astrocyte-derived exosomes after the patient undergoes the therapy; b) isolating astrocyte-derived exosomes from the first biological sample and the second biological sample; and c) detecting one or more biomarkers selected from the group consisting of complement regulatory proteins, membrane inhibitor of reactive lysis (CD59), membrane cofactor protein (CD46), decay-accelerating factor (DAF) and complement receptor type 1 (CR1) for the astrocyte-derived exosomes from the first biological sample and the second biological sample; and d) comparing the levels of the one or more biomarkers for the astrocyte-derived exosomes from the first biological sample to the levels of the one or more biomarkers for the astrocyte-derived exosomes from the second biological sample, wherein increased levels of the one or more biomarkers for the astrocyte-derived exosomes from the second biological sample compared to the levels of the one or more biomarkers for the astrocyte-derived exosomes from the first biological sample indicate that the patient is improving, and decreased levels of the one or more biomarkers for the astrocyte-derived exosomes from the second biological sample compared to the levels of the one or more biomarkers for the astrocyte-derived exosomes from the first biological sample indicate that the patient is worsening or not responding to the therapy. In some embodiments, the neurological disease is selected from the group consisting of: Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In other embodiments, the invention provides a method for monitoring a neurological disease in a subject, the method comprising: a) measuring levels of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, and C5b-C9 terminal complement complex (TCC) from astrocyte-derived exosomes from a first biological sample from the subject, wherein the first biological sample is obtained from the subject at a first time point; b) measuring levels of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, and C5b-C9 terminal complement complex (TCC) from astrocyte-derived exosomes from a second biological sample from the subject, wherein the second biological sample is obtained from the subject at a second (i.e., later) time point; and c) comparing the levels of the biomarkers for astrocyte-derived exosomes from the first biological sample to the levels of the biomarkers for astrocyte-derived exosomes from the second biological sample, wherein decreased levels of the one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, and C5b-C9 terminal complement complex (TCC) from the astrocyte-derived exosomes from the second biological sample compared to the levels of the biomarkers in the first biological sample indicate that the patient is improving, and increased levels of the one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, and C5b-C9 terminal complement complex (TCC) from the astrocyte-derived exosomes from the second biological sample compared to the levels of the biomarkers for the astrocyte-derived exosomes from the first biological sample indicate that the patient is worsening. In some embodiments, the one or more biomarkers comprises inflammatory cytokines, interleukin 6 (IL-6), tumor necrosis factor alpha (TNF-α), and interleukin 1 beta (IL-1β). In some embodiments, the one or more biomarkers comprises complement proteins, C1q, C4b, C5b, C3d, factor B, factor D, Fragment Bb, C3b, and C5b-C9 terminal complement complex (TCC). In some embodiments, the subject has a neurological disease. In other embodiments, the neurological disease is selected from the group consisting of: Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In other embodiments, the invention provides a method for monitoring a neurological disease in a subject, the method comprising: a) measuring levels of one or more biomarkers selected from the group consisting of complement regulatory proteins, membrane inhibitor of reactive lysis (CD59), membrane cofactor protein (CD46), decay-accelerating factor (DAF) and complement receptor type 1 (CR1) from astrocyte-derived exosomes from a first biological sample from the subject, wherein the first biological sample is obtained from the subject at a first time point; b) measuring levels of one or more biomarkers selected from the group consisting of complement regulatory proteins, membrane inhibitor of reactive lysis (CD59), membrane cofactor protein (CD46), decay-accelerating factor (DAF) and complement receptor type 1 (CR1) from astrocyte-derived exosomes from a second biological sample from the subject, wherein the second biological sample is obtained from the subject at a second (i.e., later) time point; and c) comparing the levels of the biomarkers for astrocyte-derived exosomes from the first biological sample to the levels of the biomarkers for astrocyte-derived exosomes from the second biological sample, wherein increased levels of the one or more biomarkers selected from the group consisting of complement regulatory proteins, membrane inhibitor of reactive lysis (CD59), membrane cofactor protein (CD46), decay-accelerating factor (DAF) and complement receptor type 1 (CR1) from the astrocyte-derived exosomes from the second biological sample compared to the levels of the biomarkers in the first biological sample indicate that the patient is improving, and decreased levels of the one or more biomarkers selected from the group consisting of complement regulatory proteins, membrane inhibitor of reactive lysis (CD59), membrane cofactor protein (CD46), decay-accelerating factor (DAF) and complement receptor type 1 (CR1) from the astrocyte-derived exosomes from the second biological sample compared to the levels of the biomarkers for the astrocyte-derived exosomes from the first biological sample indicate that the patient is worsening. In some embodiments, the one or more biomarkers comprises complement regulatory proteins, membrane inhibitor of reactive lysis (CD59), membrane cofactor protein (CD46), decay-accelerating factor (DAF) and complement receptor type 1 (CR1). In some embodiments, the subject has a neurological disease. In other embodiments, the neurological disease is selected from the group consisting of: Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In yet other embodiments, the invention provides a method of treating a patient suspected of having a neurological disease, the method comprising: a) detecting astrocyte-exosomal biomarker levels in the patient or receiving information regarding the astrocyte-exosomal biomarker levels of the patient, as determined according to a method described herein; and b) administering a therapeutically effective amount of at least one drug that alters astrocyte-exosomal biomarker levels in the subject. After treatment, the method may further comprise monitoring the response of the patient to treatment.

In other embodiments, the invention provides a method comprising: providing a biological sample from a subject suspected of having a neurological disease; detecting the presence or level of at least one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1); and administering a treatment to the subject. In one embodiment, the method further comprises administering a therapeutically effective amount of at least one drug that treats a neurological disease to the subject if increased levels of the one or more biomarkers are detected in the subject. In one embodiment, the method further comprises administering a therapeutically effective amount of at least one drug that treats a neurological disease to the subject if decreased levels of the one or more biomarkers are detected in the subject. After treatment, the method may further comprise monitoring the response of the subject to treatment. In some embodiments, the one or more biomarkers comprises inflammatory cytokines, interleukin 6 (IL-6), tumor necrosis factor alpha (TNF-α), and interleukin 1 beta (IL-1β). In some embodiments, the one or more biomarkers comprises complement proteins, C1q, C4b, C5b, C3d, factor B, factor D, Fragment Bb, C3b, and C5b-C9 terminal complement complex (TCC). In some embodiments, the one or more biomarkers comprises complement regulatory proteins, membrane inhibitor of reactive lysis (CD59), membrane cofactor protein (CD46), decay-accelerating factor (DAF) and complement receptor type 1 (CR1). In some embodiments, the neurological disease is selected from the group consisting of: Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In other embodiments, the present invention provides a method of treating a subject with a neurological disease, comprising: providing a biological sample from the subject; determining the level of at least one or more biomarkers selected from the list consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) using at least one reagent that specifically binds to said biomarkers; and prescribing a treatment regimen based on the level of the one or more biomarkers. In some embodiments, the method further comprises isolating astrocyte-derived exosomes from the biological sample. In some embodiments, the neurological disease is selected from the group consisting of: Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease. In some embodiments, the one or more biomarkers comprises inflammatory cytokines, interleukin 6 (IL-6), tumor necrosis factor alpha (TNF-α), and interleukin 1 beta (IL-1β). In some embodiments, the one or more biomarkers comprises complement proteins, C1q, C4b, C5b, C3d, factor B, factor D, Fragment Bb, C3b, and C5b-C9 terminal complement complex (TCC). In some embodiments, the one or more biomarkers comprises complement regulatory proteins, membrane inhibitor of reactive lysis (CD59), membrane cofactor protein (CD46), decay-accelerating factor (DAF) and complement receptor type 1 (CR1).

In some embodiments, the invention provides a set of biomarkers for assessing neurological disease status of a subject, the set comprising one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1), wherein astrocyte-derived exosome levels of the biomarkers in the set are assayed; and wherein the biomarker levels of the set of biomarkers determine the neurological disease status of the subject with at least 40%, 50%, 60%, 70%, 75%, 80%, 85%, 90%, 95%, 99%, or 100% specificity. In some aspects, the set of biomarkers determine the neurological disease status of the subject with at least 40%, 50%, 60%, 70%, 75%, 80%, 85%, 90%, 95%, 99%, or 100% sensitivity. In yet other aspects, the set of biomarkers determine the neurological disease status of the subject with at least 40%, 50%, 60%, 70%, 75%, 80%, 85%, 90%, 95%, 99%, or 100% accuracy. In some embodiments, the one or more biomarkers comprises inflammatory cytokines, interleukin 6 (IL-6), tumor necrosis factor alpha (TNF-α), and interleukin 1 beta (IL-1β). In some embodiments, the one or more biomarkers comprises complement proteins, C1q, C4b, C5b, C3d, factor B, factor D, Fragment Bb, C3b, and C5b-C9 terminal complement complex (TCC). In some embodiments, the one or more biomarkers comprises complement regulatory proteins, membrane inhibitor of reactive lysis (CD59), membrane cofactor protein (CD46), decay-accelerating factor (DAF) and complement receptor type 1 (CR1).

In other embodiments, the invention provides a composition comprising at least one in vitro complex comprising a labeled antibody bound to a biomarker protein selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1), wherein said biomarker protein is extracted from astrocyte-derived exosomes of a subject who has been diagnosed with a neurological disease, suspected of having a neurological disease, or at risk of developing a neurological disease. The antibody may be detectably labeled with any type of label, including, but not limited to, a fluorescent label, an enzyme label, a chemiluminescent label, or an isotopic label. In some embodiments, the composition is in a detection device (i.e., device capable of detecting labeled antibody). In some embodiments, the one or more biomarkers comprises inflammatory cytokines, interleukin 6 (IL-6), tumor necrosis factor alpha (TNF-α), and interleukin 1 beta (IL-1β). In some embodiments, the one or more biomarkers comprises complement proteins, C1q, C4b, C5b, C3d, factor B, factor D, Fragment Bb, C3b, and C5b-C9 terminal complement complex (TCC). In some embodiments, the one or more biomarkers comprises complement regulatory proteins, membrane inhibitor of reactive lysis (CD59), membrane cofactor protein (CD46), decay-accelerating factor (DAF) and complement receptor type 1 (CR1). In some embodiments, the neurological disease is selected from the group consisting of: Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In other embodiments, the invention provides a kit for detecting or monitoring a neurological disease in a subject. In some embodiments, the kit may include a container for holding a biological sample isolated from a subject who has been diagnosed or suspected of having a neurological disease or at risk of developing a neurological disease, at least one agent that specifically detects a biomarker of the present invention; and printed instructions for reacting the agent with astrocyte-derived exosomes from the biological sample or a portion of the biological sample to detect the presence or amount of at least one biomarker. In other embodiments, the kit may also comprise one or more agents that specifically bind astrocyte-derived exosomes for use in isolating astrocyte-derived exosomes from a biological sample. In yet other embodiments, the kit may further comprise one or more control reference samples and reagents for performing an immunoassay. In certain embodiments, the agents may be packaged in separate containers. In some embodiments, the kit comprises agents for measuring the levels of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1). In yet other embodiments, the kit further comprises an antibody that binds to an astrocyte-derived exosome surface marker (e.g., Glutamine Aspartate Transporter (GLAST)).

In other embodiments, the invention provides a method for treating a neurological disease, the method comprising the steps of: providing a biological sample from a subject suspected of having a neurological disease, wherein the sample comprises astrocyte-derived exosomes; measuring the level of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) from the biological sample, wherein an altered level of the one or more biomarkers in the sample relative to the level in a control sample is indicative of a need for treatment; and administering an effective amount of an agent to the subject thereby treating the neurological disease in the subject. In some embodiments, the agent is a recombinant complement control protein selected from the group consisting of recombinant membrane inhibitor of reactive lysis (CD59), recombinant membrane cofactor protein (CD46), recombinant decay-accelerating factor (DAF) and recombinant complement receptor type 1 (CR1). In other embodiments, the agent is neutralizing monoclonal antibodies to effector complement components or their receptors, decoy complement receptors or receptor antagonists, or an esterase inhibitor.

These and other embodiments of the present invention will readily occur to those of skill in the art in light of the disclosure herein, and all such embodiments are specifically contemplated.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIGS. 1A-1C set forth data showing astrocyte-derived exosomal levels of inflammatory cytokines in plasma of patients with Alzheimer's disease (AD) relative to those of matched controls without Alzheimer's disease (Control). Each point represents the value for a control subject or AD patient and the horizontal line in point clusters is the mean level for that group. The significance of differences between values for controls and AD patients was calculated by an unpaired Student's t test; ⁺=p<0.01 and *=p<0.001.

FIGS. 2A-2H set forth data showing altered levels of astrocyte-derived exosomal complement protein biomarkers in plasma of patients with Alzheimer's disease relative to those of matched controls without Alzheimer's disease (Control). Each point represents the value for a control subject or AD patient and the horizontal line in point clusters is the mean level for that group. The significance of differences between values for controls and AD patients was calculated by an unpaired Student's t test; **=p<0.0001.

FIGS. 3A-3D set forth data showing decreased levels of astrocyte-derived exosomal complement system regulatory proteins in plasma of patients with Alzheimer's disease (AD) and matched controls without Alzheimer's disease (Control). Each point represents the value for a control subject or AD patient and the horizontal line in point clusters is the mean level for that group. The significance of differences between values for controls and AD patients was calculated by an unpaired Student's t test; *=p<0.01 and **=p<0.0001.

FIGS. 4A-4H set forth data showing plasma levels of complement proteins and cytokines in astrocyte-derived exosomes from subjects in a longitudinal study. Study subjects included subjects with Alzheimer's disease who had provided blood at two times: first when cognitively intact (AD1) and again five to 12 years later after diagnosis of dementia (AD2) and cognitively normal controls (Control), who were age- and gender-matched with the AD1 group. Each point represents the value for a control subject or AD patient and the horizontal line in point clusters is the mean level for that group. The significance of differences between values for controls and AD1 patients was calculated by an unpaired Student's t test and for differences between values for AD1 and AD2 patients was calculated by a paired Student's t test **=p<0.0001.

FIGS. 5A-4H set forth data showing ADE levels of complement effector proteins in cross-sectional control, MCI and AD groups. Each point represents the value for a control subject or patient and the horizontal line in point clusters is the mean level for that group. Mean±S.E.M. for control, stable MCI (MCIS), MCI that converted to dementia (MCIC) and AD patient values, respectively, are 14,560±1423, 8,845±744, 51,274±3706 and 46,135±3001 pg/ml for C1q, 66,936±5660, 98,464±13,248, 698,662±57,983 and 162,747±8076 pg/ml for C4b, 1478±171, 1539±200, 5763±653 pg/ml and 5875±816 pg/ml for factor D, 106,508±13,449, 22,503±2908, 144,103±13,792 and 245,094±15,609 pg/ml for factor B fragment Bb, 3105±198, 3109±319, 5937±478 and 5238±244 pg/ml for C5b, 25,571±2412, 7453±876, 64,389±4593 and 78,256±5499 pg/ml for C3b, 358±35.0, 523±47.2, 1117±117 and 1187±134 pg/ml for C5b-C9 TCC, and 1130±131, 1122±110, 1123±116 and 1157±134 pg/ml for MBL. The significance of differences between values for controls and AD patients, and between values for MCIS and MCIC patients was calculated by an unpaired Student's t test and shown over the last two bars of each set; *=p<0.01 and **=p<0.0001.

FIGS. 6A-4D set forth data showing ADE levels of complement regulatory proteins in cross-sectional control, MCI and AD groups. Each point represents the value for a control subject or patient and the horizontal line in point clusters is the mean level for that group. Mean±S.E.M. for control, MCIS, MCIC and AD patient values, respectively, are 35,166±3981, 11,486±1581, 2796±230 and 3825±499 pg/ml for DAF, 62.4±5.48, 245±37.3, 47.1±4.78 and 35.3±2.91 pg/ml for CD46, 473±38.2, 475±51.9, 268±29.9 and 286±20.6 pg/ml for CR1, and 1268±84.1, 865±103, 584±60.6 and 353±38.5 pg/ml for CD59. The significance of differences between values for controls and AD patients, and between values for MCIS and MCIC patients was calculated by an unpaired Student's t test and shown over the last two bars of each set; ⁺=p<0.05, *=p<0.01 and **=p<0.0001.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items. It must be noted that as used herein and in the appended claims, the singular forms “a,” “an,” and “the” include plural references unless context clearly dictates otherwise. Thus, for example, a reference to “a fragment” includes a plurality of such fragments, a reference to an “antibody” is a reference to one or more antibodies and to equivalents thereof known to those skilled in the art, and so forth.

### DESCRIPTION OF THE INVENTION

It is to be understood that the invention is not limited to the particular methodologies, protocols, cell lines, assays, and reagents described herein, as these may vary. It is also to be understood that the terminology used herein is intended to describe particular embodiments of the present invention, and is in no way intended to limit the scope of the present invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

The present invention relates, in part, to the discovery that astrocyte-derived exosomal biomarkers can be used to detect pathogenesis of neurological diseases, including Alzheimer's disease. The inventor has demonstrated that astrocyte-derived exosome (ADE) levels of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) are altered in subjects with neurological disease (see, e.g., Example 1).

The present invention also provides compositions for use in the methods described herein. Such compositions may include small molecule compounds; peptides and proteins including antibodies or functionally active fragments thereof.

The present invention further provides kits for identifying a subject at risk of a neurological disease or prescribing a therapeutic regimen or predicting benefit from therapy in a subject having a neurological disease or at risk of developing a neurological disease. In these embodiments, the kits comprise one or more antibodies which specifically bind astrocyte-derived exosomes, one or more antibodies which specifically bind an astrocyte-derived exosomal biomarker of the present invention, one or more containers for collecting and or holding the biological sample, and instructions for the kits use.

The section headings are used herein for organizational purposes only, and are not to be construed as in any way limiting the subject matter described herein.

### Complement System

The complement system provides an early acting mechanism to initiate and amplify the inflammatory response to microbial infection and other acute insults. While complement activation provides a valuable first-line defense against potential pathogens, the activities of complement that promote a protective inflammatory response can also represent a potential threat to the host. For example, C3 and C5 proteolytic products recruit and activate neutrophils. These activated cells are indiscriminate in their release of destructive enzymes and may cause organ damage. In addition, complement activation may directly cause the deposition of lytic complement components, such as C5b-C9 TCC, on nearby host cells as well as on microbial targets, resulting in host cell lysis. Some products of complement activation, such as C3b, bind to neurons as well as microbes and thereby cause attachment of neuron-destructive CNS cells, such as microglia.

Complement can be activated through either of two distinct enzymatic cascades, referred to as the classical and alternative pathways. The classical pathway is usually triggered by antibody bound to a foreign particle and thus requires prior exposure to that particle for the generation of specific antibody. There are four plasma proteins specifically involved in the classical pathway: C1, C2, C4 and C3. The interaction of C1 with the Fc regions of IgG or IgM in immune complexes activates a C1 protease that can cleave plasma protein C4, resulting in the C4a and C4b fragments. C4b can bind another plasma protein, C2. The resulting species, C4b2, is cleaved by the C1 protease to form the classical pathway C3 convertase, C4b2a. Addition of the C3 cleavage product, C3b, to C3 convertase leads to the formation of the classical pathway C5 convertase, C4b2a3b.

In contrast to the classical pathway, the alternative pathway is spontaneously triggered by foreign or other abnormal surfaces (bacteria, yeast, virally infected cells, or damaged tissue) and is therefore capable of an immediate response to an invading organism. There are four plasma proteins directly involved in the alternative pathway: C3, factors B and D, and properdin (also called factor P). The initial interaction that triggers the alternative pathway is not completely understood. However, it is thought that spontaneously activated C3 (C3b) binds factor B, which is then cleaved by factor D to form the complex C3bBb that possesses C3 convertase activity. The resulting convertase proteolytically modifies additional C3, producing the C3b fragment, which can covalently attach to the target and then interact with factors B and D and form the alternative pathway C3 convertase, C3bBb. The alternative pathway C3 convertase is stabilized by the binding of properdin. However, properdin binding is not required to form a functioning alternative pathway C3 convertase. Since the substrate for the alternative pathway C3 convertase is C3, C3 is therefore both a component and a product of the reaction. As the C3 convertase generates increasing amounts of C3b, an amplification loop is established. In as much as the classical pathway also may generate C3b that can bind factor B, both pathways may amplify activation of the alternative pathway. This allows more C3b to deposit on a target. For example, as described above, the binding of antibody to antigen initiates the classical pathway. If antibodies latch on to bacteria, the classical pathway generates C3b, which couples to target pathogens. However, it has been suggested that from 10% to 90% of the subsequent C3b deposited may come from the alternative pathway. The actual contribution of the alternative pathway to the formation of additional C3b subsequent to classical pathway initiation has not been clearly quantified and thus remains unknown. Addition of C3b to the C3 convertase leads to the formation of the alternative pathway C5 convertase, C3bBbC3b.

Both the classical and alternative pathways involve C3b and converge at C5, which is cleaved to form products with multiple proinflammatory effects. The converged pathway has been referred to as the terminal complement pathway. C5a is the most potent anaphylatoxin, inducing alterations in smooth muscle and vascular tone, as well as vascular permeability. It is also a powerful chemotaxin and activator of both neutrophils and monocytes. C5a-mediated cellular activation can significantly amplify inflammatory responses by inducing the release of multiple additional inflammatory mediators, including cytokines, hydrolytic enzymes, arachidonic acid metabolites and reactive oxygen species. C5 cleavage leads to the formation of C5b-9, also known as the membrane terminal attack complex (MAC or TCC). There is now strong evidence that MAC may play an important role in inflammation in addition to its role as a lytic pore-forming complex. The present invention provides methods for detecting astrocyte-derived exosomal levels of complement system proteins. The administration of one or more neutralizing monoclonal antibodies to effector complement components or their receptors, decoy complement receptors or receptor antagonists, and esterase inhibitors of complement mediator generation may suppress ongoing complement-mediated neuronal injury and be useful for treating neurological disease. In some embodiments, the methods of the present invention are used to treat a neurological disease in a subject. In other embodiments, the present invention provides a method for treating a subject having a neurological disease, comprising the steps of: providing a biological sample from a subject suspected of having a neurological disease, wherein the sample comprises astrocyte-derived exosomes; measuring the level of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) from the biological sample, wherein an altered level of the one or more biomarkers in the sample relative to the level in a control sample is indicative of a need for treatment; and administering an effective amount of neutralizing monoclonal antibodies to effector complement components or their receptors, decoy complement receptors or receptor antagonists, or esterase inhibitors of complement mediator generation to the subject thereby treating the neurological disease in the subject. In certain embodiments, the neurological disease is Alzheimer's disease.

Many regulatory proteins exist to prevent excessive complement activation and to protect our cells and tissues from damage. The complement system distinguishes self from non-self via a range of specialized cell-surface and soluble proteins. These proteins belong to a family called the regulators of complement activation (RCA) or complement control proteins (CCP). Complement control proteins (or complement regulatory proteins) work in concert to maintain activation of the complement system at a level optimal for host defenses against microbes without damaging host tissues. Many of the complement control proteins act on the convertases, C3b.Bb and C4b.2a, which are bimolecular complexes formed early on in the complement cascade, but CD59 blocks formation of C5b-C9 TCC.

Every cell in the human body is protected by one or more of the membrane-associated RCA proteins, CR1, DAF or MCP. Factor H and C4BP circulate in the plasma and are recruited to self-surfaces through binding to host-specific polysaccharides such as the glycosaminoglycans. Most act to disrupt the formation of the convertases or to shorten the life-span of any complexes that do manage to form. Their presence on self-surfaces, and their absence from the surfaces of foreign particles, means that these regulators perform the important task of targeting complement to where it is needed—on the invading bacterium for example—while preventing activation on host tissues. For example, C3b.Bb is an important convertase that is part of the alternative pathway, and it is formed when factor B binds C3b and is subsequently cleaved. To prevent this from happening, factor H competes with factor B to bind C3b; if it manages to bind, then the convertase is not formed. Factor H can bind C3b much more easily in the presence of sialic acid, which is a component of most cells in the human body; conversely, in the absence of sialic acid, factor B can bind C3b more easily. This means that if C3b is bound to a “self” cell, the presence of sialic acid and the binding of factor H will prevent the complement cascade from activating; if C3b is bound to a bacterium, factor B will bind and the cascade will be set off as normal. The present invention provides methods for detecting astrocyte-derived exosomal levels of complement regulatory proteins. The administration of one or more recombinant complement control proteins early in neurological disease, guided by their levels in astrocyte-derived exosomes of subjects, could limit recruitment of complement mechanisms preventatively. In some embodiments, the methods of the present invention are used to treat a neurological disease in a subject. In other embodiments, the present invention provides a method for treating a subject having a neurological disease, comprising the steps of: providing a biological sample from a subject suspected of having a neurological disease, wherein the sample comprises astrocyte-derived exosomes; measuring the level of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) from the biological sample, wherein an altered level of the one or more biomarkers in the sample relative to the level in a control sample is indicative of a need for treatment; and administering an effective amount of an recombinant complement control protein to the subject thereby treating the neurological disease in the subject. In certain embodiments, the neurological disease is Alzheimer's disease. In other embodiments, the recombinant complement control protein is selected from the group consisting of recombinant membrane inhibitor of reactive lysis (CD59), recombinant membrane cofactor protein (CD46), recombinant decay-accelerating factor (DAF) and recombinant complement receptor type 1 (CR1).

### Biological Sample

The present invention provides biomarkers and diagnostic and prognostic methods for neurological diseases. Biomarker are detected from astrocyte-derived exosomes from a biological sample obtained from a subject. Biological samples can include any bodily fluid comprising exosomes, including, but not limited to, whole blood, plasma, serum, lymph, amniotic fluid, and umbilical cord blood.

In some embodiments, the biological sample of the invention can be obtained from blood. In some embodiments, about 1-10 mL of blood is drawn from a subject. In other embodiments, about 10-50 mL of blood is drawn from a subject. Blood can be drawn from any suitable area of the body, including an arm, a leg, or blood accessible through a central venous catheter. In some embodiments, blood is collected following a treatment or activity. For example, blood can be collected following a medical exam. The timing of collection can also be coordinated to increase the number and/or composition of astrocyte-derived exosomes present in the sample. For example, blood can be collected following exercise or a treatment that induces vascular dilation.

Blood may be combined with various components following collection to preserve or prepare samples for subsequent techniques. For example, in some embodiments, blood is treated with an anticoagulant, a cell fixative, a protease inhibitor, a phosphatase inhibitor, or preservative(s) for protein or DNA or RNA following collection. In some embodiments, blood is collected via venipuncture using a needle and a syringe that is emptied into collection tubes containing an anticoagulant such as EDTA, heparin, or acid citrate dextrose (ACD). Blood can also be collected using a heparin-coated syringe and hypodermic needle. Blood can also be combined with components that will be useful for cell culture. For example, in some embodiments, blood is combined with cell culture media or supplemented cell culture media (e.g., cytokines). In certain embodiments, platelet-rich plasma (PRP) is mixed with PBS to block ex vivo platelet activation before centrifugation to yield platelet-poor plasma (PPP).

### Enrichment or Isolation of Astrocyte-Derived Exosomes

Samples can be enriched for astrocyte-derived exosomes through positive selection, negative selection, or a combination of positive and negative selection. In some embodiments, exosomes are directly captured. In other embodiments, blood cells are captured and exosomes are collected from the remaining biological sample.

Samples can also be enriched for exosomes based on the biochemical properties of exosomes. The first step is physical isolation entailing polymer precipitation with centrifugation in one or two cycles. Then, for example, samples can be enriched for exosomes based on differences in antigens. In some of the embodiments, antibody-conjugated magnetic or paramagnetic beads in magnetic field gradients or fluorescently labeled antibodies with flow cytometry are used. In some of the embodiments based on metabolic differences, dye uptake/exclusion measured by flow cytometry or another sorting technology is used. Samples can also be enriched for exosomes based on other biochemical properties known in the art. For example, samples can be enriched for exosomes using ligands or soluble receptors.

In some embodiments, surface markers are used to positively enrich astrocyte-derived exosomes in the sample. In other embodiments, cell surface markers that are not found on exosomes are used to negatively enrich exosomes by depleting cell populations. Modified versions of flow cytometry sorting may also be used to further enrich for astrocyte-derived exosomes using surface markers or intracellular or extracellular markers conjugated to fluorescent labels. Intracellular and extracellular markers may include nuclear stains or antibodies against intracellular or extracellular proteins preferentially expressed in exosomes. Cell surface markers may include cell surface antigens that are preferentially expressed on astrocyte-derived exosomes. In some embodiments, the cell surface marker is an astrocyte-derived exosome surface marker, including, for example, Glutamine Aspartate Transporter (GLAST). In some embodiments, a monoclonal antibody that specifically binds to GLAST (e.g., ACSA-1, mouse anti-human GLAST antibody) is used to enrich or isolate astrocyte-derived exosomes from the sample. In certain aspects, the antibody against GLAST is biotinylated. In this embodiment, the biotinylated antibody can form an antibody-exosome complex that can be subsequently isolated using streptavidin-agarose resin or beads. In other embodiments, the antibody is a monoclonal anti-human GLAST antibody (e.g., ACSA-1).

In other embodiments, astrocyte-derived exosomes are isolated or enriched from a biological sample comprising: contacting a biological sample with an agent under conditions wherein an astrocyte-derived exosome present in said biological sample binds to said agent to form an astrocyte-agent complex; and isolating said exosome from said exosome-agent complex to obtain a sample containing said exosome, wherein the purity of the exosomes present in the sample is greater than the purity of exosomes present in the biological sample. In certain embodiments, the contacting is incubating or reacting. In certain embodiments, the exosomes are astrocyte-derived exosomes. In certain embodiments, the agent is an antibody or a lectin. Lectins useful for forming an exosome-lectin complex are described in U.S. Patent Application Publication No. 2012/0077263. In some embodiments, multiple isolating or enriching steps are performed. In certain aspects of the present embodiment, a first isolating step is performed to isolate exosomes from a blood sample freed of plasma membrane-derived membrane vesicles and a second isolating step is performed to isolate astrocyte-derived exosomes from other exosomes. In other embodiments, the exosome portion of the exosome-agent complex is lysed using a lysis reagent and the protein levels of the lysed exosome are assayed. In some embodiments, the antibody-exosome complex is created on a solid phase. In yet other embodiments, the methods further comprise releasing the exosome from the antibody-exosome complex. In certain embodiments, the solid phase is non-magnetic beads, magnetic beads, agarose, or sepharose. In other embodiments, the vesicle is released by exposing the antibody-exosome complex to low pH between 3.5 and 1.5. In yet other embodiments, the released exosome is neutralized by adding a high pH solution. In other embodiments, the released exosomes are lysed by incubating the released exosomes with a lysis solution. In still other embodiments, the lysis solution contains inhibitors for proteases and phosphatases.

### Neurological Diseases

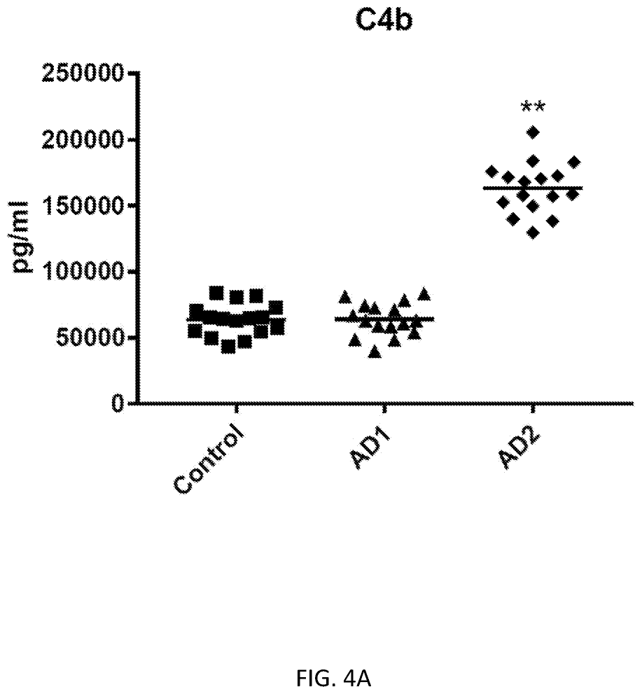

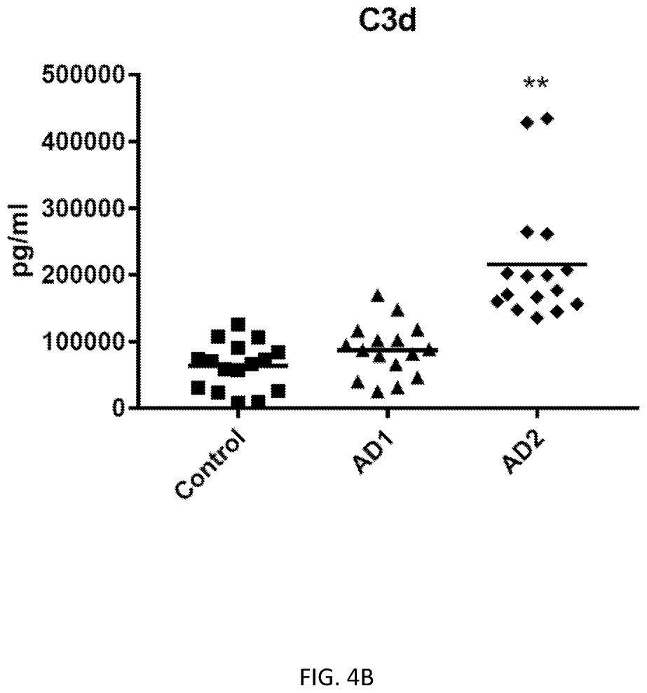

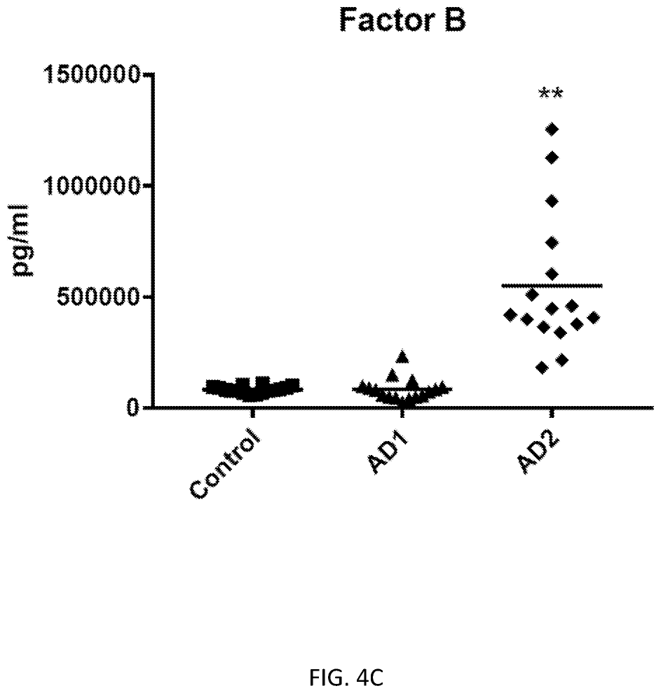

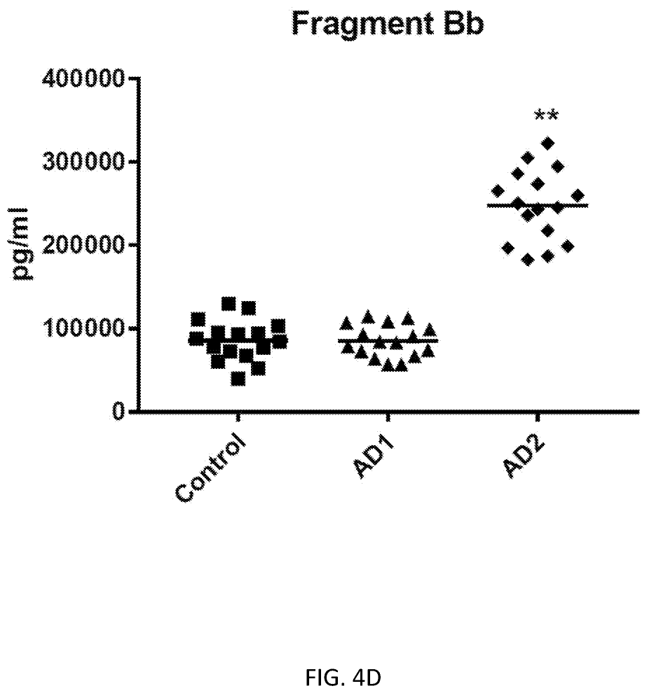

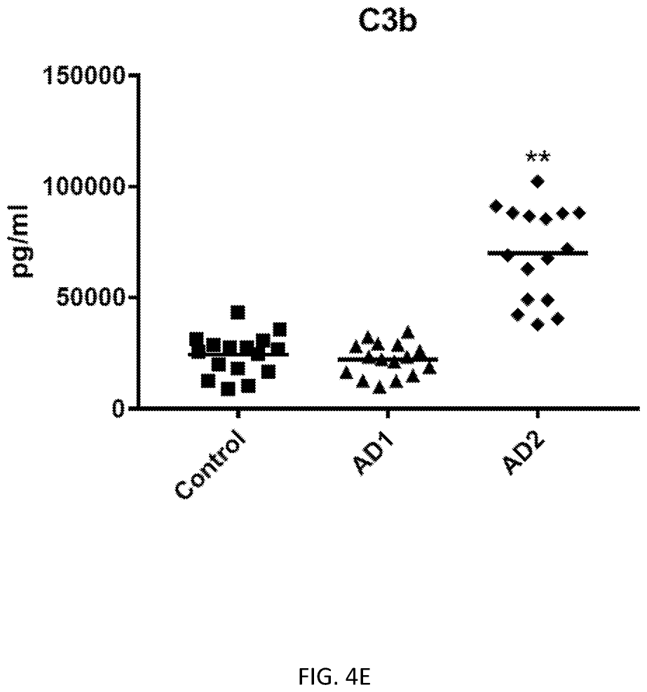

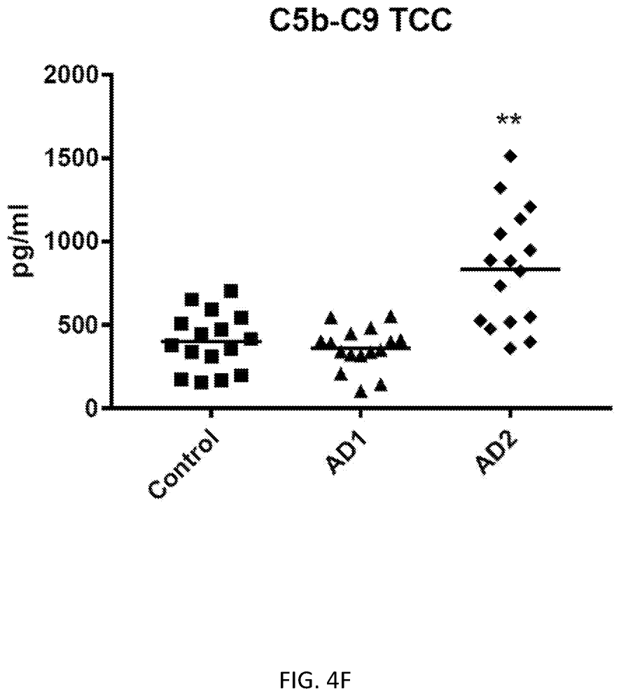

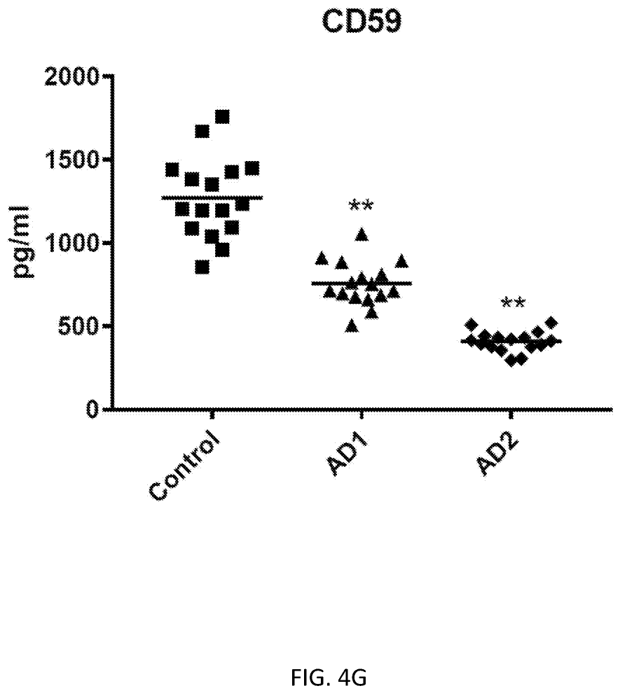

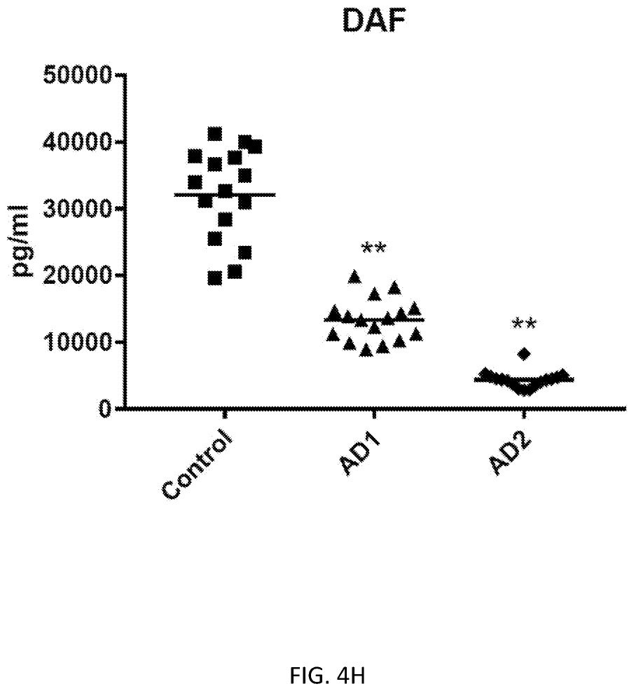

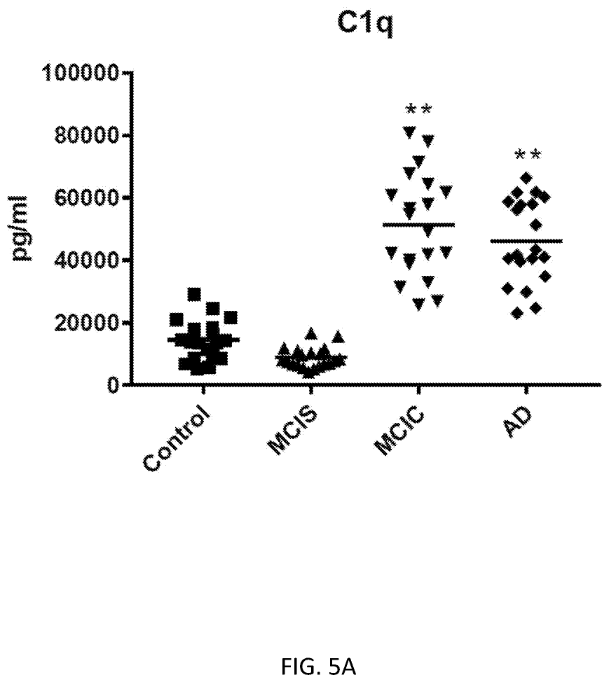

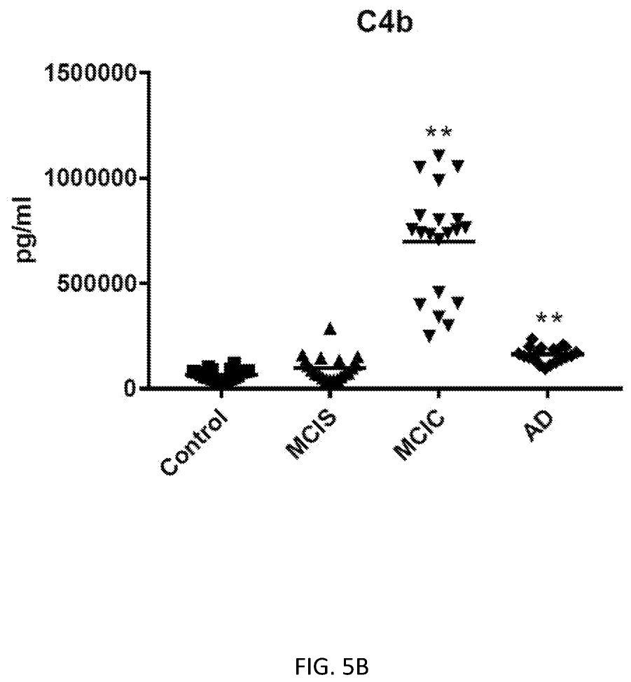

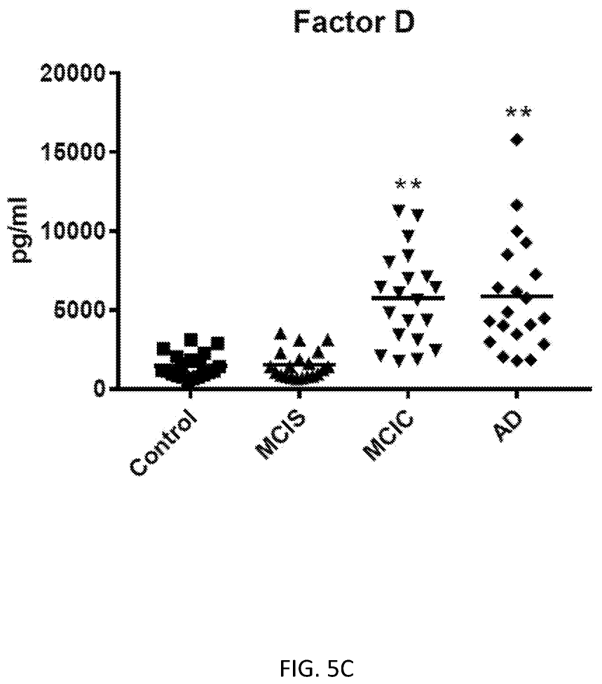

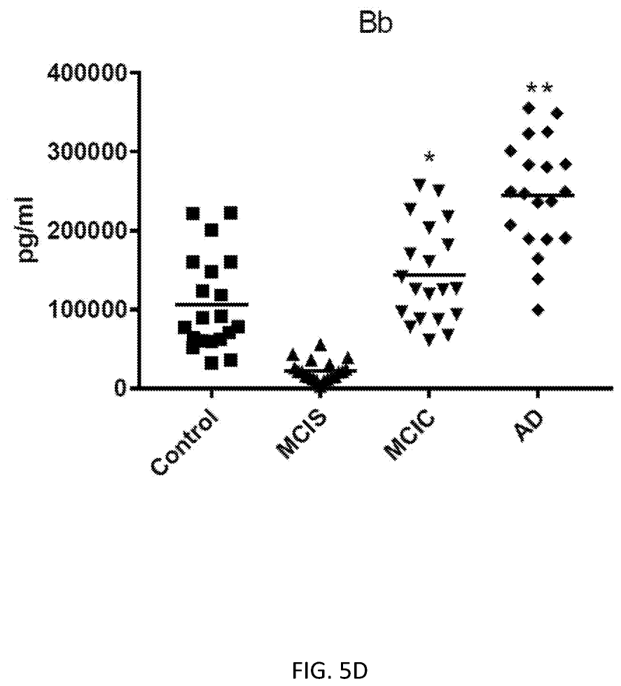

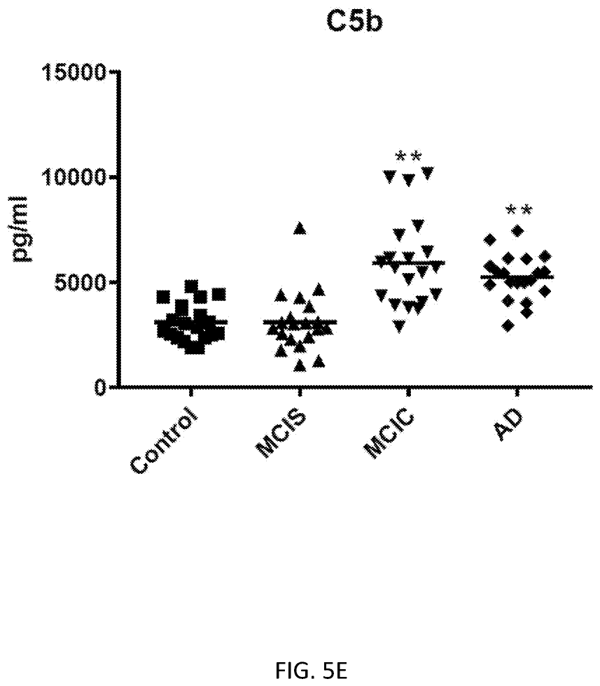

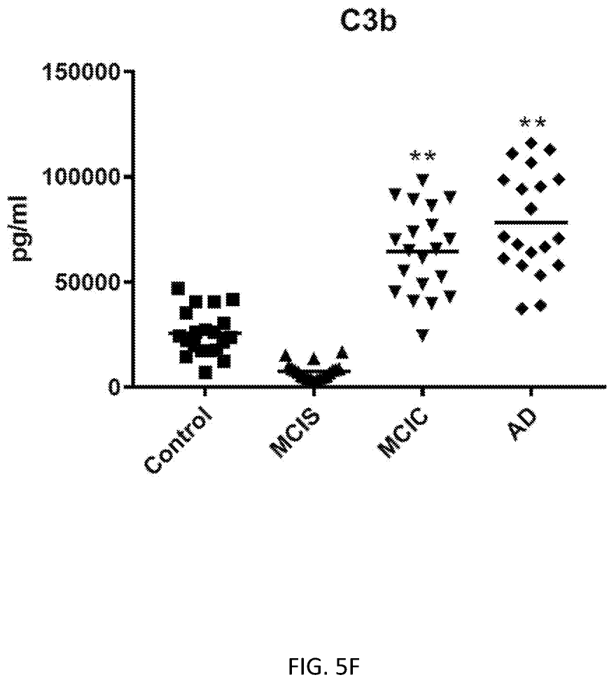

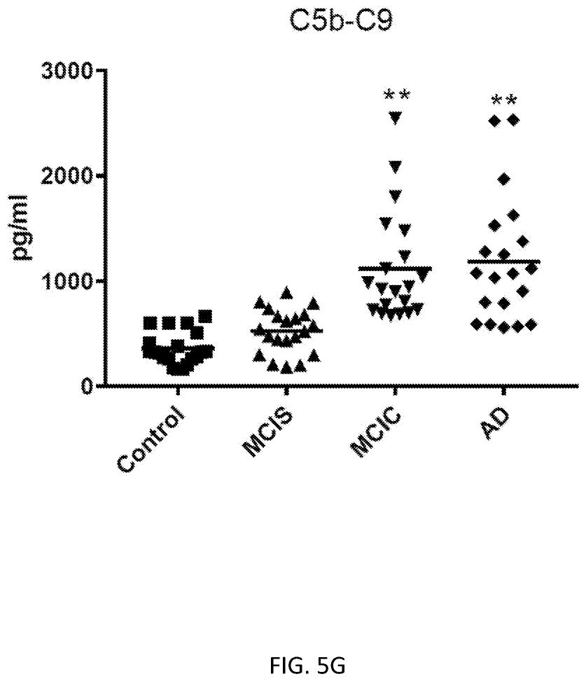

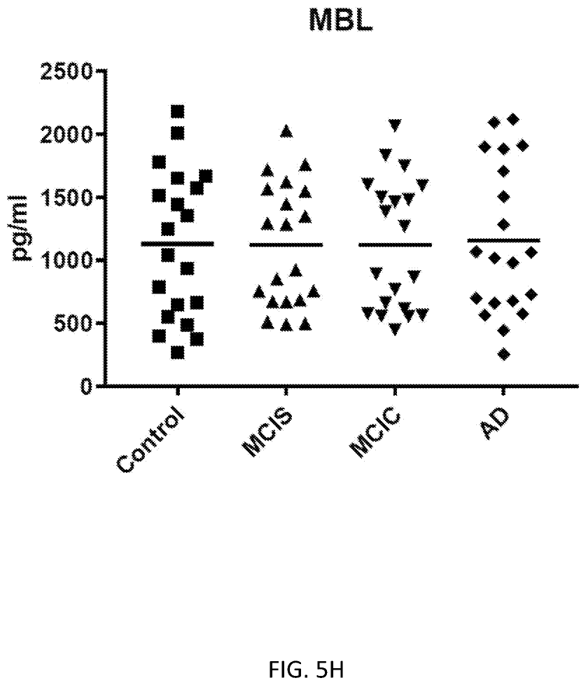

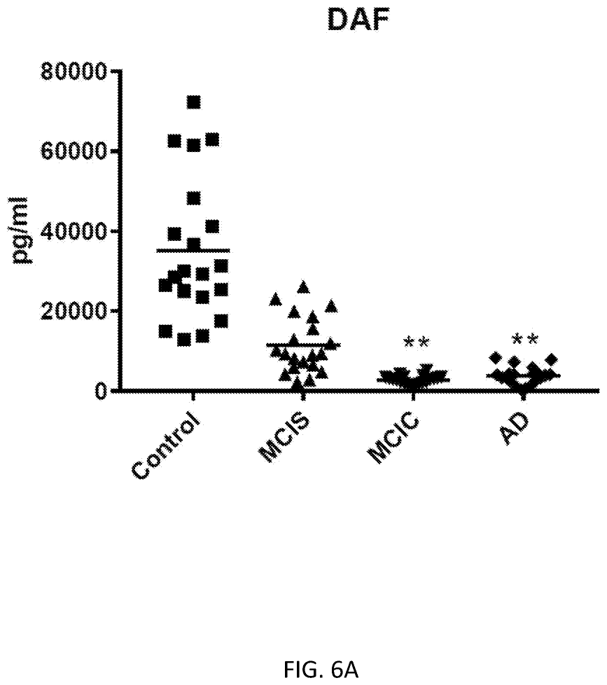

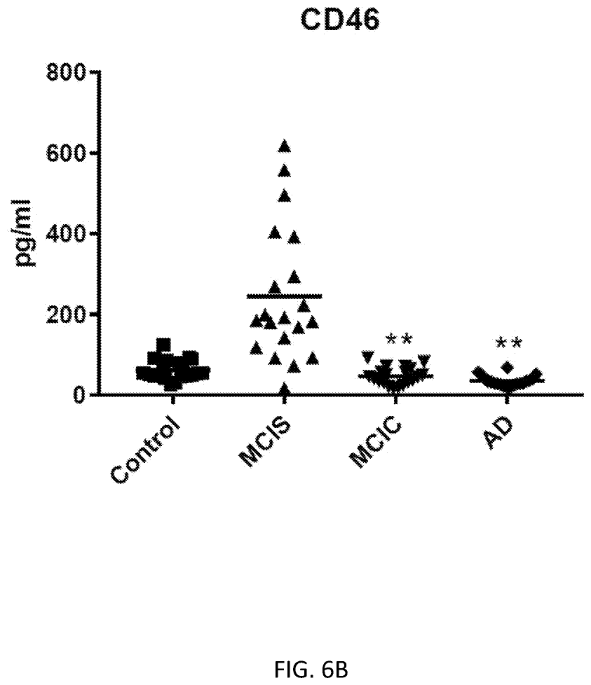

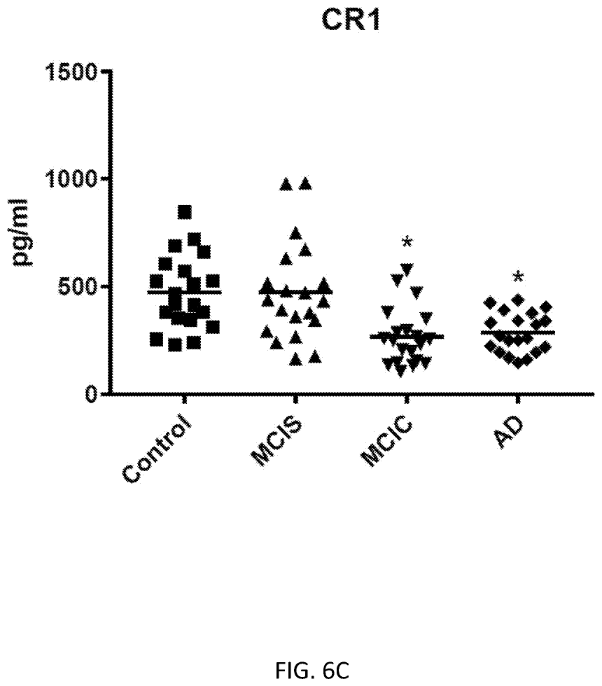

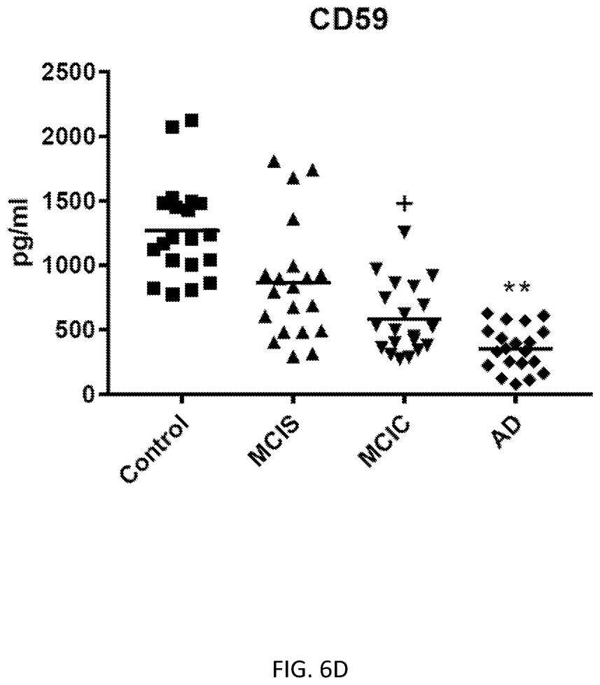

The present invention provides methods for diagnosing a neurological disease in a subject and/or identifying a subject at risk of developing a neurological disease, or prescribing a therapeutic regimen or predicting benefit from therapy. elevated ADE levels of neurotoxic complement effector proteins in AD patients compared to matched control subjects suggest that damage to neurons inflicted by type A1 reactive astrocytes also may be mediated by products of the classical and alternative complement systems. Diminished ADE levels of four membrane-associated complement pathway regulatory proteins in AD relative to those in control subjects were found at the preclinical AD1 stage of AD, when complement effector proteins were still at control levels (FIG. 4). This time-course for development of complement abnormalities suggests that inadequate control is the primary underlying mechanism of enhanced complement activation in type A1 reactive astrocytes. Hence, astrocyte-derived exosomal biomarker abnormalities are associated with development or worsening of a neurological disease (e.g., Alzheimer's disease). Accordingly, detection of astrocyte-derived exosomal biomarker abnormalities can be used to identify individuals who will benefit from therapy.

In some embodiments, the neurological disease is selected from the group consisting of: Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In certain embodiments, the subject is a mammalian subject, including, e.g., a cat, a dog, a rodent, etc. In preferred embodiments, the subject is a human subject.

In some embodiments, the present invention enables a medical practitioner to diagnose or prognose one or more neurological diseases in a subject. In yet other embodiments, the present invention enables a medical practitioner to identify a subject at risk of developing a neurological disease. In other embodiments, the present invention enables a medical practitioner to predict whether a subject will later develop a neurological disease, such as, for example, Alzheimer's disease. In further embodiments the present invention enables a medical practitioner to prescribe a therapeutic regimen or predict benefit from therapy in a subject having a neurological disease or at risk of developing a neurological disease. For example, the administration of one or more recombinant complement control proteins early in Alzheimer's disease, guided by their levels in astrocyte-derived exosomes of individual patients, could limit recruitment of complement mechanisms preventatively. In later phases of Alzheimer's disease, when complement activation has appeared, neutralizing monoclonal antibodies to effector complement components or their receptors, decoy complement receptors or receptor antagonists, and esterase inhibitors of complement mediator generation may suppress ongoing complement-mediated neuronal injury.

### Biomarkers

Astrocyte-derived exosomal cargo levels of biomarker proteins are assayed for a subject having or at-risk of having a neurological disease. In some embodiments, one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) are assayed in order to detect whether or not a subject has a neurological disease. In one embodiment, all of the biomarkers, inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1), are assayed in combination to detect a neurological disease. In some embodiments, one or more biomarkers selected from the group consisting of inflammatory cytokines, interleukin 6 (IL-6), tumor necrosis factor alpha (TNF-α), and interleukin 1 beta (IL-1β), are assayed in combination to detect a neurological disease. In some embodiments, one or more biomarkers selected from the group consisting of complement proteins, C1q, C4b, C5b, C3d, factor B, factor D, Fragment Bb, C3b, and C5b-C9 terminal complement complex (TCC), are assayed in combination to detect a neurological disease. In some embodiments, one or more biomarkers selected from the group consisting of complement regulatory proteins, membrane inhibitor of reactive lysis (CD59), membrane cofactor protein (CD46), decay-accelerating factor (DAF) and complement receptor type 1 (CR1), are assayed in combination to detect a neurological disease. In some embodiments, the one or more biomarkers are assayed in the preclinical phase.

One of ordinary skill in the art has several methods and devices available for the detection and analysis of the biomarkers of the instant invention. With regard to polypeptides or proteins in patient test samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassay (RIAs), competitive binding assays, planar waveguide technology, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the biomarkers is also contemplated by the present invention. The antibodies could be immobilized onto a variety of solid supports, such as magnetic or chromatographic matrix particles, the surface of an assay place (such as microtiter wells), pieces of a solid substrate material (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of biomarkers may be carried out separately or simultaneously with one test sample. Several biomarkers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in biomarker levels, as well as the absence of change in biomarker levels, would provide useful information about disease status that includes, but is not limited to the appropriateness of drug therapies, the effectiveness of various therapies, identification of the severity of a neurological disease, susceptibility to neurological disease, and prognosis of the patient's outcome, including risk of development of a neurological disease, such as, for example, risk of developing Alzheimer's disease.

An assay consisting of a combination of the biomarkers referenced in the instant invention may be constructed to provide relevant information related to differential diagnosis. Such a panel may be constructed using 1, 2, 3, 4, 5, 6, 7, 8, 9, 10, 15, 20, or more individual markers. The analysis of a single biomarker or subsets of biomarkers comprising a larger panel of biomarkers could be carried out using methods described within the instant invention to optimize clinical sensitivity or specificity in various clinical settings.

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings. Particularly useful physical formats comprise surfaces having a plurality of discrete, addressable locations for the detection of a plurality of different analytes. Such formats include protein microarrays, or “protein chips” and capillary devices.

Biomarkers of the present invention serve an important role in the early detection and monitoring of neurological disease (e.g., Alzheimer's disease). Biomarkers are typically substances found in a bodily sample that can be measured. The measured amount can correlate with underlying disorder or disease pathophysiology and probability of developing a neurological disease in the future. In patients receiving treatment for their condition, the measured amount will also correlate with responsiveness to therapy.

In some embodiments, the biomarker is measured by a method selected from the group consisting of immunohistochemistry, immunocytochemistry, immunofluorescence, immunoprecipitation, western blotting, and ELISA.

### Clinical Assay Performance

The methods of the present invention for detecting neurological disease may be used in clinical assays to diagnose or prognose a neurological disease in a subject, identify a subject at risk of a neurological disease (e.g., Alzheimer's disease), and/or for prescribing a therapeutic regimen or predicting benefit from therapy in a subject having a neurological disease. Clinical assay performance can be assessed by determining the assay's sensitivity, specificity, area under the ROC curve (AUC), accuracy, positive predictive value (PPV), and negative predictive value (NPV). Disclosed herein are assays for diagnosing or prognosing a neurological disease in a subject, identifying a subject at risk of a neurological disease, or for prescribing a therapeutic regimen or predicting benefit from therapy in a subject having a neurological disease.

The clinical performance of the assay may be based on sensitivity. The sensitivity of an assay of the present invention may be at least about 40%, 45%, 50%, 55%, 60%, 65%, 70%, 75%, 80%, 85%, 90%, 95%, 99%, or 100%. The clinical performance of the assay may be based on specificity. The specificity of an assay of the present invention may be at least about 40%, 45%, 50%, 55%, 60%, 65%, 70%, 75%, 80%, 85%, 90%, 95%, 99%, or 100%. The clinical performance of the assay may be based on area under the ROC curve (AUC). The AUC of an assay of the present invention may be at least about 0.5, 0.55, 0.6, 0.65, 0.7, 0.75, 0.8, 0.85, 0.9, or 0.95. The clinical performance of the assay may be based on accuracy. The accuracy of an assay of the present invention may be at least about 40%, 45%, 50%, 55%, 60%, 65%, 70%, 75%, 80%, 85%, 90%, 95%, 99%, or 100%.

### Compositions

Compositions useful in the methods of the present invention include compositions that specifically recognize one or more astrocyte-derived exosomal biomarkers associated with neurological disease, including inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1), or any combination thereof. In some embodiments, the composition enhances the activity of at least one biomarker. In other embodiments, the composition decreases the activity of at least one biomarker. In some embodiments, the composition increases the levels of at least one biomarker in the subject. In other embodiments, the composition decreases the levels of at least one biomarker in the subject. In yet other embodiments, the composition comprises a peptide, a nucleic acid, an antibody, or a small molecule.

In certain embodiments, the present invention relates to compositions that specifically detect a biomarker associated with neurological disease. As detailed elsewhere herein, the present invention is based upon the finding that astrocyte-derived exosomal inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) proteins are specific biomarkers for neurological disease. In one embodiment, the compositions of the invention specifically bind to and detect one or more of the biomarkers inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1), or any combination thereof. The composition of the present invention can comprise an antibody, a peptide, a small molecule, a nucleic acid, and the like.

In some embodiments, the composition comprises an antibody, wherein the antibody specifically binds to a biomarker or astrocyte-derived exosomes. The term “antibody” as used herein and further discussed below is intended to include fragments thereof which are also specifically reactive with a biomarker or vesicle (e.g., exosome). Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. Antigen-binding portions may also be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), single domain antibodies, bispecific antibodies, chimeric antibodies, humanized antibodies, diabodies and polypeptides that contain at least a portion of an immunoglobulin that is sufficient to confer specific antigen binding to the polypeptide. In certain embodiments, the antibody further comprises a label attached thereto and able to be detected (e.g., the label can be a radioisotope, fluorescent compound, enzyme or enzyme co-factor).

In certain embodiments, an antibody of the invention is a monoclonal antibody, and in certain embodiments, the invention makes available methods for generating novel antibodies that specifically bind the biomarker or the exosome of the invention. For example, a method for generating a monoclonal antibody that specifically binds a biomarker or exosome, may comprise administering to a mouse an amount of an immunogenic composition comprising the biomarker or exosome, or fragment thereof, effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the biomarker or exosome. Once obtained, a hybridoma can be propagated in a cell culture, optionally in culture conditions where the hybridoma-derived cells produce the monoclonal antibody that binds specifically to the biomarker or exosome. The monoclonal antibody may be purified from the cell culture.

The term “specifically reactive with” or “specifically binds” as used in reference to an antibody is intended to mean, as is generally understood in the art, that the antibody is sufficiently selective between the antigen of interest (e.g., a biomarker or exosome) and other antigens that are not of interest. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less.

Antibodies can be generated to bind specifically to an epitope of an astrocyte-derived exosome or a biomarker of the present invention, including, for example, astrocyte-derived exosome surface markers, such as Glutamine Aspartate Transporter (GLAST).

In addition, the techniques used to screen antibodies in order to identify a desirable antibody may influence the properties of the antibody obtained. A variety of different techniques are available for testing interaction between antibodies and antigens to identify particularly desirable antibodies. Such techniques include ELISAs, surface plasmon resonance binding assays (e.g., the Biacore binding assay, Biacore AB, Uppsala, Sweden), sandwich assays (e.g., the paramagnetic bead system of IGEN International, Inc., Gaithersburg, Md.), western blots, immunoprecipitation assays, immunocytochemistry, and immunohistochemistry.

In some embodiments, the present invention relates to compositions used for treating or preventing a neurological disease. As detailed elsewhere herein, abnormal levels of astrocyte-derived exosomal biomarkers are implicated in the pathology of neurological disease, including Alzheimer's disease. Therefore, in one embodiment, the present invention provides compositions that inhibit or reduce abnormalities in levels of astrocyte-derived exosomal biomarkers. Compositions useful for preventing and/or reducing abnormalities in levels of astrocyte-derived exosomal biomarkers may include proteins, peptides, nucleic acids, small molecules, and the like.

### Methods of Treatment

The present invention provides methods of treating a neurological disease associated with astrocyte-derived exosomal abnormalities in a subject, comprising administering to the subject an effective amount of a composition, wherein the composition inhibits astrocyte-derived exosomal abnormalities.

Furthermore, the methods of the invention can be used for monitoring the efficacy of therapy in a patient. The method comprises: analyzing the levels of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) from astrocyte-derived exosomes from biological samples from the patient before and after the patient undergoes the therapy, in conjunction with respective reference levels for the biomarkers. Increasing levels of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, and C5b-C9 terminal complement complex (TCC) astrocyte-derived exosomal biomarkers correlate with increased neurological disease severity and indicate that the patient is worsening or not responding to the therapy, and decreasing levels of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, and C5b-C9 terminal complement complex (TCC) astrocyte-derived exosomal biomarkers correlate with reduced neurological disease severity and indicate that the condition of the patient is improving (e.g., lower risk of Alzheimer's disease or dementia).

Decreasing levels of complement regulatory proteins, membrane inhibitor of reactive lysis (CD59), membrane cofactor protein (CD46), decay-accelerating factor (DAF) and complement receptor type 1 (CR1) astrocyte-derived exosomal biomarkers correlate with increased neurological disease severity and indicate that the patient is worsening or not responding to the therapy, and increasing levels of complement regulatory proteins, membrane inhibitor of reactive lysis (CD59), membrane cofactor protein (CD46), decay-accelerating factor (DAF) and complement receptor type 1 (CR1) astrocyte-derived exosomal biomarkers correlate with reduced neurological disease severity and indicate that the condition of the patient is improving (e.g., lower risk of Alzheimer's disease or dementia).

In some embodiments, the methods of the invention provide a method for treating neurological disease the method comprising the steps of: obtaining a biological sample from a subject suspected of having a neurological disease, wherein the sample comprises astrocyte-derived exosomes; measuring the level of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) from the biological sample, wherein an altered level of the one or more biomarkers in the sample relative to the level in a control sample is indicative of a need for treatment; and administering an effective amount of an agent to the subject thereby treating the neurological disease in the subject.

### Kits

Another aspect of the invention encompasses kits for detecting or monitoring neurological disease in a subject. A variety of kits having different components are contemplated by the current invention. Generally speaking, the kit will include the means for quantifying one or more biomarkers in a subject. In another embodiment, the kit will include means for collecting a biological sample, means for quantifying one or more biomarkers in the biological sample, and instructions for use of the kit contents. In certain embodiments, the kit comprises a means for enriching or isolating astrocyte-derived exosomes in a biological sample. In further aspects, the means for enriching or isolating astrocyte-derived exosomes comprises reagents necessary to enrich or isolate astrocyte-derived exosomes from a biological sample. In certain aspects, the kit comprises a means for quantifying the amount of a biomarker. In further aspects, the means for quantifying the amount of a biomarker comprises reagents necessary to detect the amount of a biomarker.

These and other embodiments of the present invention will readily occur to those of ordinary skill in the art in view of the disclosure herein.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications are intended to fall within the scope of the appended claims.

### Example 1

### Biomarker Detection and Levels in Astrocyte-Derived Exosomes from Humans with Neurological Disease

Biomarkers were detected and measured in astrocyte-derived exosomes from subjects with neurological disease as follows. Blood samples were obtained from 28 patients with early Alzheimer's disease (AD), mild cognitive impairment (MCI), or mild dementia and 28 age- and gender-matched control subjects.

Ten milliliters of venous blood were drawn into 0.5 ml saline with EDTA, incubated for 10 minutes at room temperature, and centrifuged for 15 minutes at 2500×g. Plasmas were stored in 0.25-ml aliquots at −80° C. CSF levels of P-T181-tau and Aβ 1-42 were quantified by xMAP Technology (Luminex Corp., Austin, Tex., USA) using Inno-Bia AlzBio3 kits (Innogenetics, Ghent, Belgium).

Aliquots of 0.25 ml plasma were incubated with 0.1 ml thromboplastin D (Thermo Fisher Scientific, Waltham, Mass., USA), followed by addition of 0.15 ml of calcium- and magnesium-free Dulbecco's balanced salt solution with protease inhibitor cocktail (Roche, Indianapolis, Ind., USA) and phosphatase inhibitor cocktail (Thermo Fisher Scientific) (DBS⁺⁺) (8). After centrifugation at 3000×g for 30 min at 4° C., total exosomes were harvested from resultant supernatants by 126 uL per tube of ExoQuick (System Biosciences, Mountain View, Calif., USA) precipitation. To enrich ADEs, total exosomes were resuspended in 0.35 ml of DBS⁺⁺ and incubated for 60 min at room temperature with 1.5 μg of mouse anti-human Glutamine Aspartate Transporter (GLAST) (ACSA-1) biotinylated antibody (Miltenyi Biotec, Inc., Auburn, Calif.) in 50 μL of 3% BSA (1:3.33 dilution of Blocker BSA 10% solution in DBS⁻² [Thermo Scientific, Inc.]) per tube with mixing, followed by addition of 10 μl of streptavidin-agarose Ultralink resin (Thermo Scientific, Inc.) in 40 μL of 3% BSA and incubation for 30 min at room temperature with mixing. After centrifugation at 600×g for 10 min at 4° C. and removal of the supernate, each pellet was suspended in 100 μL of 0.05 M glycine-HCl (pH 3.0) by gentle mixing for 10 sec and centrifuged at 4,000×g for 10 min, all at 4° C. Supernatants then were transferred to clean tubes containing 25 μL of 10% BSA and 10 μL of 1 M Tris-HCl (pH=8.0) and mixed before addition of 365 μL of mammalian protein extraction reagent (M-PER) (ThermoFisher Scientific). Resultant 0.5 ml lysates of ADEs were stored at −80° C. NDEs were prepared as described in Goetzl et al. (2016) Faseb J 30, 4141-4148.

ADE and NDE proteins were quantified by ELISA kits for human tetraspanning exosome marker CD81, complement fragment C4b, complement fragment C5b, DAF (CD55) (American Research Products-Cusabio, Waltham, Mass., USA), glutamine synthetase (GluSyn), CR1 (American Research Products-Cloud-Clone Corp., Waltham, Mass.), glial fibrillary acidic protein (GFAP) (EMD-Millipore Corp., Billerica, Mass.), complement component C3d, CD46 (LifeSpan Biosciences, Inc., Seattle, Wash.), complement fragment C3b, complement factor B, C1q portion of the Cl complement component (Abcam, Inc., Cambridge, Mass.), Bb fragment of complement factor B (Quidel-Microvue, San Diego, Calif.), terminal complement complex C5b-C9 (Elabscience, Bethesda, Md.), CD59, interleukin-1-beta (IL-1β) (Ray Biotech, Inc., Norcross, Ga.), tumor necrosis factor-alpha (TNF-α), complement factor D and IL-6 (ThermoFisher-Invitrogen, LaFayette, Colo.). The mean value for all determinations of CD81 in each assay group was set at 1.00 and relative values of CD81 for each sample were used to normalize their recovery.

A Shapiro-Wilks test showed that data in all sets were distributed normally. Statistical significance of differences between means for cross-sectional groups AD and C, and between longitudinal groups AD1 and C were determined with an unpaired Student's t test, including a Bonferroni correction, and the significance of differences between means for longitudinal groups AD1 and AD2 were determined with a paired Student's t test (Prism 6; GraphPad Software, La Jolla, Calif., USA).

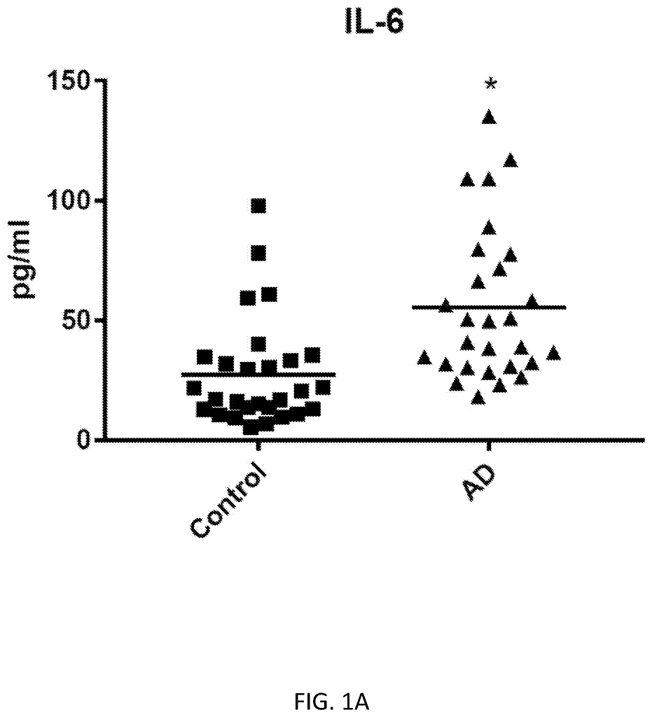

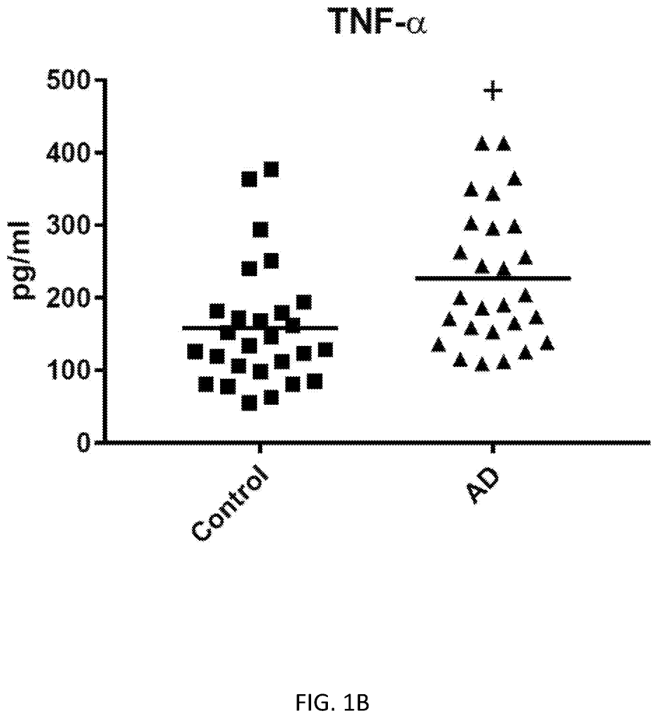

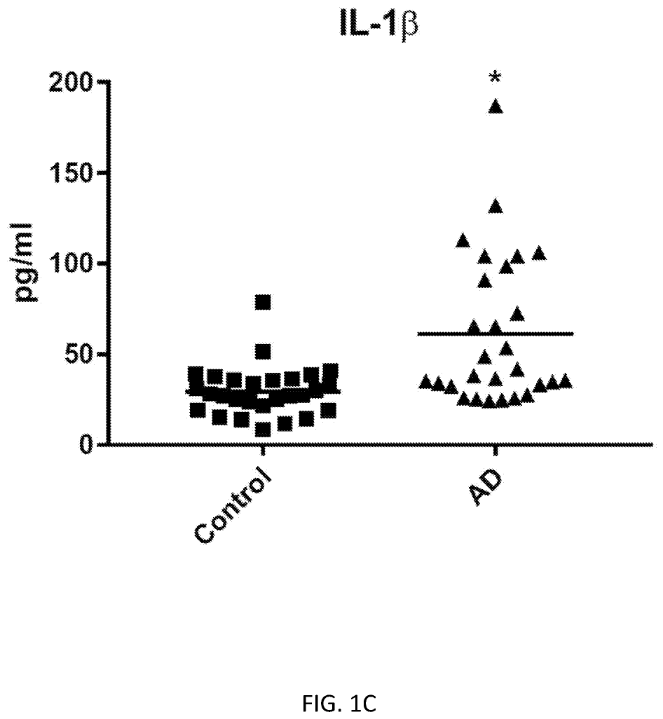

As shown in FIGS. 1A-1C, the mean CD81-normalized ADE levels of IL-6, TNF-α and IL-1β all were significantly higher for AD patients than age- and gender-matched controls, but there was extensive overlap between the sets of values for AD patients and controls (See FIGS. 1A-1C). Mean±SEM control and AD patient values, respectively, were 27.4±4.22 pg/ml and 55.5±6.02 pg/ml for IL-6, 158±16.2 pg/ml and 226±17.9 pg/ml for TNF-α, and 29.6±2.63 pg/ml and 61.3±7.75 pg/ml for IL-1β.

To contrast quantities of the cytokine protein cargoes of ADEs with those of NDEs, NDEs were harvested immunochemically from the same precipitated populations of total plasma exosomes as the ADEs for ten of the AD patients and their matched controls. CD81-normalized NDE values of TNF-α were 20.8±2.15 pg/ml (mean±S.E.M.) for AD patients and 26.3±2.47 pg/ml for controls, respectively, that were significantly lower than those of ADEs. Cytokine protein levels of IL-6 and IL-1β were not reliably detected in any of the NDE samples.

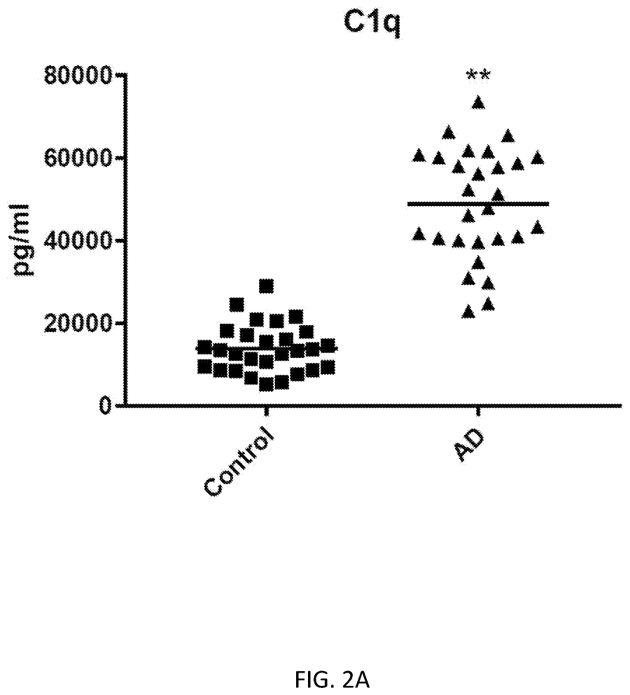

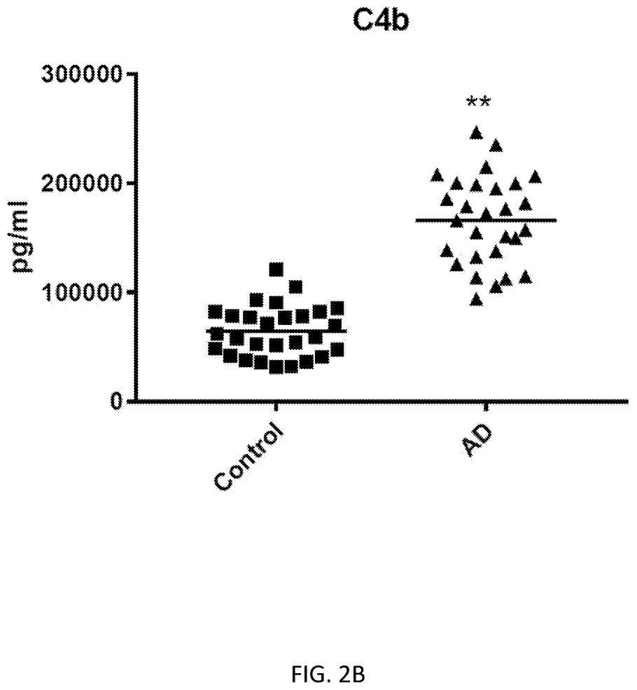

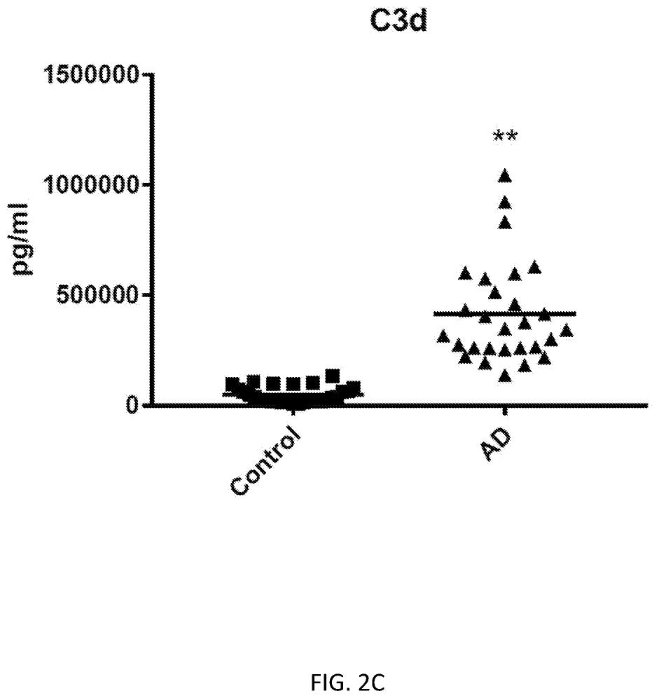

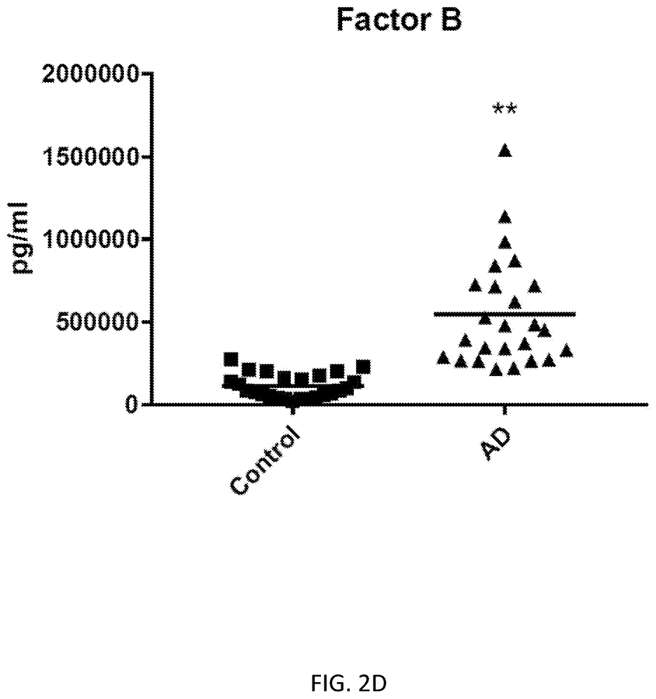

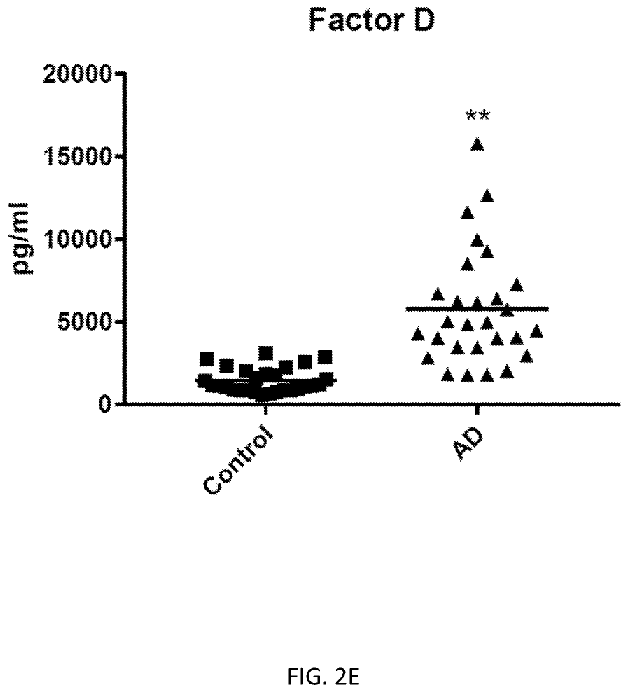

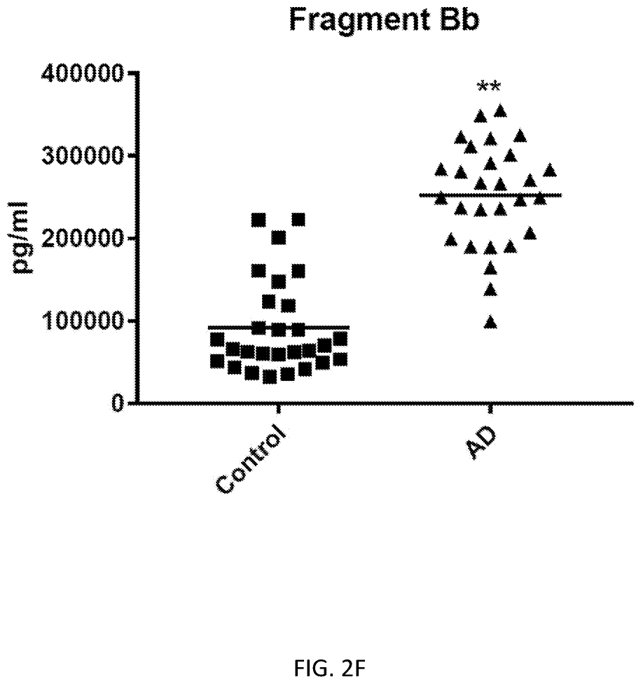

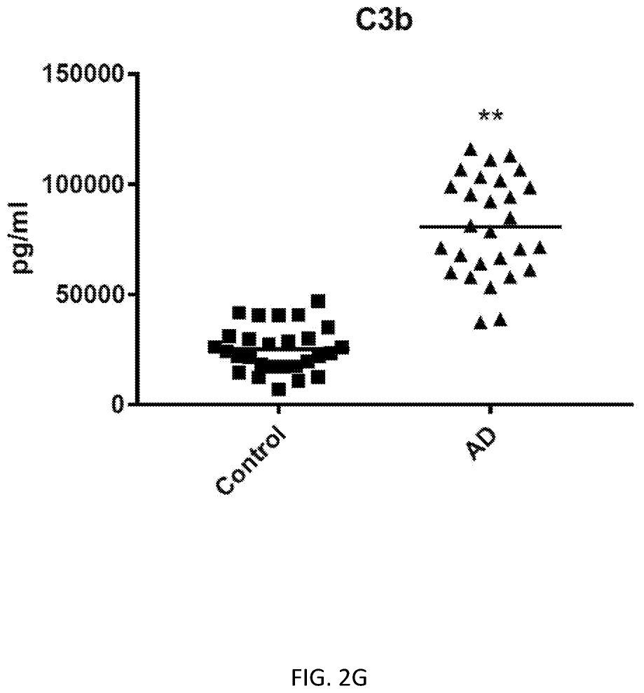

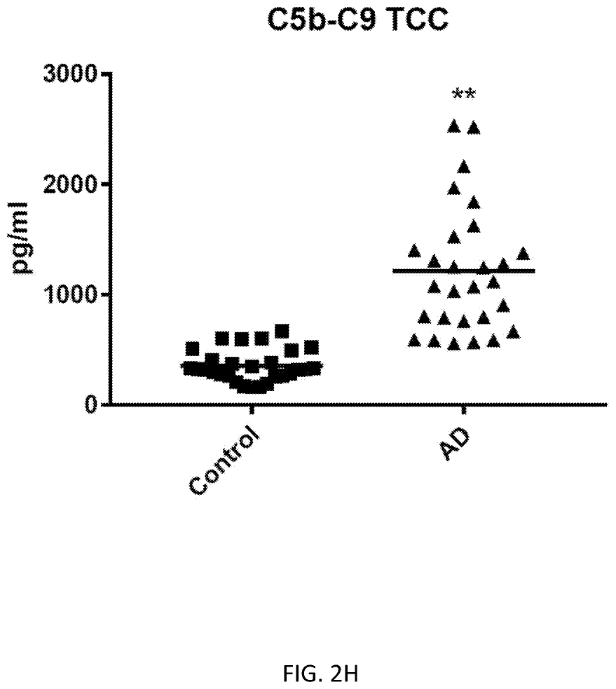

As shown in FIGS. 2A-2H, ADE levels of components, fragments and complexes of the complement systems all were substantially higher than those of the immune cytokines assessed (see FIGS. 2A-2H). CD81-normalized mean ADE levels of components exclusively of the classical pathway of complement, C1q and C4b (FIGS. 2A-2B), and exclusively of the alternative pathway of complement, factor B, factor D and fragment Bb were significantly higher for AD patients than controls (p<0.0001) (see FIGS. 2D-2F). CD81-normalized mean ADE values of the complement fragments C3b and C3d (FIGS. 2C and 2G), and of the C5b-C9 terminal complement complex (TCC) (FIG. 2H) generated by both pathways also were significantly higher for AD patients than controls (p<0.0001). There was far less overlap between values of the AD patients and controls for the complement protein analytes than the cytokines. Mean±S.E.M. control and AD patient values, respectively, were 13,902±1105 pg/ml and 48,906±2528 for C1q, 64,305±4408 pg/ml and 166,151±7647 pg/ml for C4b, 48,705±6,966 pg/ml and 416,093±44,866 pg/ml for C3d, 113,321±14,402 pg/ml and 547,292±66,082 pg/ml for complement factor B, 1465±140 pg/ml and 5800±658 pg/ml for complement factor D, 91,933±10,641 pg/ml and 252,271±11,912 pg/ml for factor B fragment Bb, 25,175+1973 pg/ml and 80,703+4286 pg/ml for C3b, and 355+27.1 pg/ml and 1212±109 pg/ml for C5b-C9 TCC.

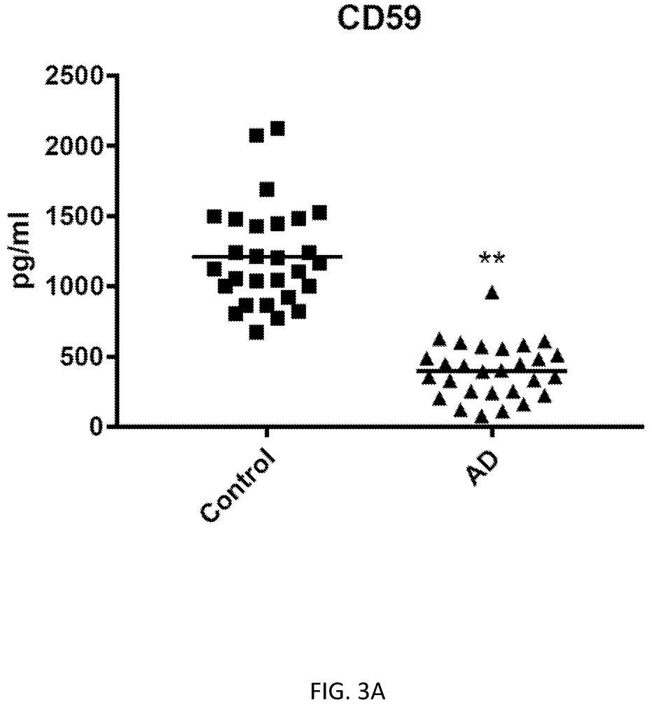

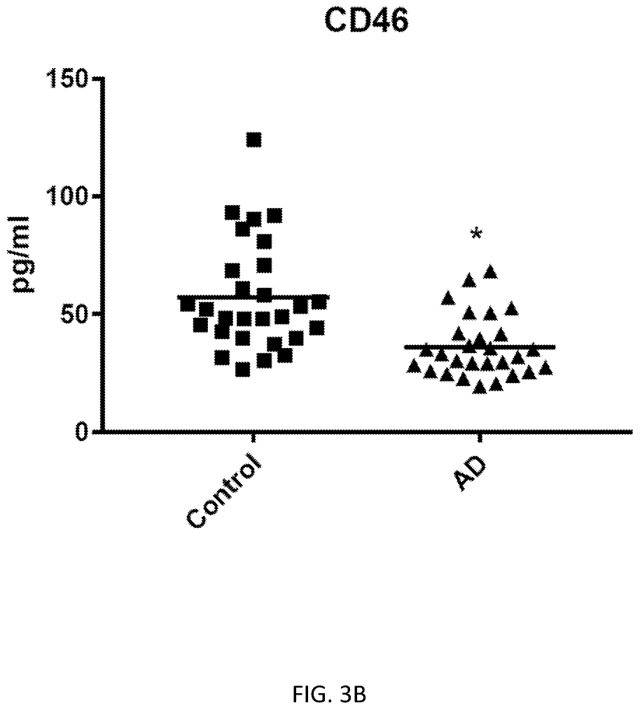

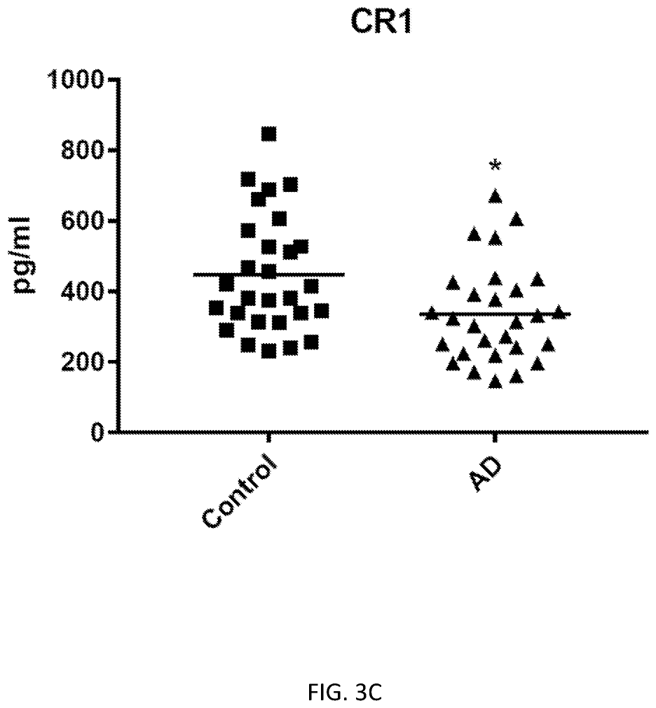

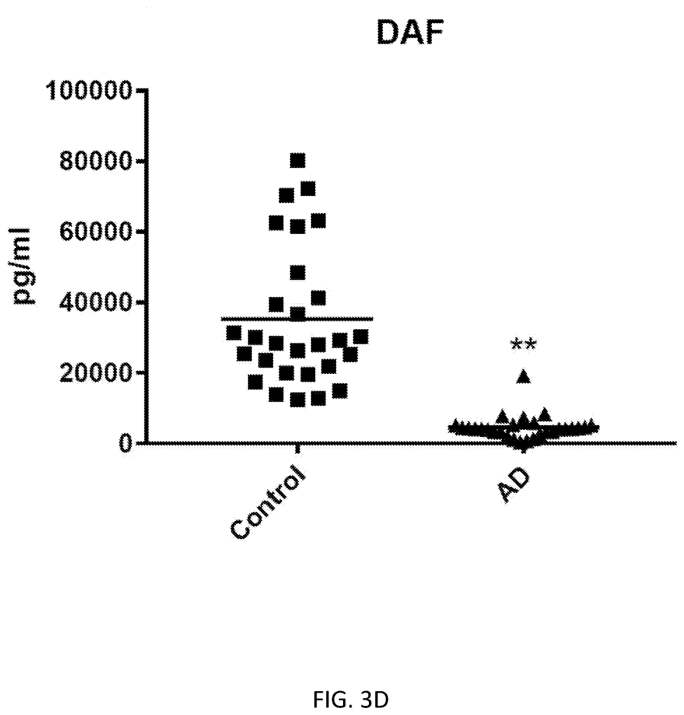

As shown in FIGS. 3A-3D, CD81-normalized mean ADE levels of all four membrane-associated complement system regulatory proteins were significantly lower for AD patients than controls (p<0.01 for CR1, p=0.0003 for CD46, and p<0.0001 for CD59 and DAF) (see FIGS. 3A-3D). Mean±S.E.M. control and AD patient values, respectively, were 1211±68.5 pg/ml and 398±37.1 pg/ml for CD59, 57.2±4.39 pg/ml and 36.1±2.47 pg/ml for CD46, 447±31.4 pg/ml and 336±26.1 pg/ml for CR1, and 35,197±3735 pg/ml and 4,563±654 pg/ml for DAF.

These results showed that ADE levels of IL-6, TNF-α, IL1β, C1q, C4b, C3d, factor B, factor D, Bb, C3b and C5b-C9 terminal complement complex (TCC) are increased in subjects with neurological disease. The results also showed that ADE levels of complement regulatory proteins, CD59, CD46, CR1, and DAF are decreased in subjects with neurological disease. These results demonstrated that the methods of the present invention are useful for detecting biomarkers and measuring biomarker protein levels in astrocyte-derived exosomes. These results further demonstrated that the methods of the present invention may be used to detect exosomal complement biomarkers associated with pathogenesis of neurological diseases, including Alzheimer's disease. These results further showed that methods of the present invention are useful for prognosis, diagnosis, treating or monitoring treatment of exosomal complement abnormalities associated with neurological diseases. The results suggested that the methods of the present invention would be useful for treating neurological diseases.

### Example 2

### Longitudinal Study of Biomarker Detection and Levels in Astrocyte-Derived Exosomes from Subjects that Developed Neurological Disease

A longitudinal study was performed to detect and measure levels of various biomarkers of the present invention in astrocyte-derived exosomes (ADE) from subjects before and after the development of neurological disease as follows. Blood samples were obtained from 16 patients with moderate AD who had provided blood at two times: first when cognitively intact (AD1, Table 1b) and again five to 12 years later after diagnosis of dementia (AD2, Table 1b). Plasma samples from 16 cognitively normal controls, who were age- and gender-matched with the AD1 group, were obtained in the same time period.

Blood samples from the subjects were processed and astrocyte-derived exosomes were isolated as described above in Example 1. ADE proteins were quantified using ELISA kits for human tetraspanning exosome marker CD81, complement fragment C4b, DAF (CD55) (American Research Products-Cusabio, Waltham, Mass., USA), glutamine synthetase (GluSyn), CR1 (American Research Products-Cloud-Clone Corp., Waltham, Mass.), glial fibrillary acidic protein (GFAP) (EMD-Millipore Corp., Billerica, Mass.), complement component C3d, CD46 (LifeSpan Biosciences, Inc., Seattle, Wash.), complement fragment C3b, complement factor B, C1q portion of the C1 complement component (Abcam, Inc., Cambridge, Mass.), Bb fragment of complement factor B (Quidel-Microvue, San Diego, Calif.), terminal complement complex C5b-C9 (Elabscience, Bethesda, Md.), CD59, interleukin-1-beta (IL-1β) (Ray Biotech, Inc., Norcross, Ga.), tumor necrosis factor-alpha (TNF-α), complement factor D and IL-6 (ThermoFisher-Invitrogen, LaFayette, Colo.). The mean value for all determinations of CD81 in each assay group was set at 1.00 and relative values of CD81 for each sample were used to normalize their recovery.

A Shapiro-Wilks test showed that data in all sets were distributed normally. Statistical significance of differences between means for cross-sectional groups AD and C, and between longitudinal groups AD1 and C were determined with an unpaired Student's t test, including a Bonferroni correction, and the significance of differences between means for longitudinal groups AD1 and AD2 were determined with a paired Student's t test (Prism 6; GraphPad Software, La Jolla, Calif., USA).

TABLE 1
Characteristics of Alzheimer's Disease Subjects and Control Subjects

| | | | | | |
|---|---|---|---|---|---|
| | Total | Male/ | Ages | MMSE | ADAS-cog |
| Diagnosis | Number | Female | Mean ± S.E.M. | Mean ± S.E.M. | Mean ± S.E.M |

a. Cross-Sectional Sets

| | | | | | |
|---|---|---|---|---|---|
| C | 28 | 12/16 | 73.2 ± 1.47 | 29.7 ± 0.13 | 3.32 ± 0.31 |
| AD | 28 | 12/16 | 73.1 ± 1.44 | 25.6 ± 0.83* | 13.7 ± 1.31* |

b. Longitudinal Sets

| | | | | | |
|---|---|---|---|---|---|
| C | 16 | 9/7 | 69.7 ± 2.04 | 29.1 ± 0.87 | 3.42 ± 0.58 |
| AD₁ | 16 | 9/7 | 70.3 ± 1.75 | 28.5 ± 0.62 | 3.94 ± 0.60 |
| AD₂ | 16 | 9/7 | 79.1 ± 1.83 | 21.6 ± 1.46* | 19.8 ± 1.89* |

AD and C are the patients and controls in the cross-sectional study of Alzheimer's disease (AD). AD₁ and AD₂ are the groups of AD patients evaluated at two times in the longitudinal study, at a preclinical phase and after conversion to moderate dementia, respectively, and C here are the controls matched to the AD₁ patients.
MMSE = Mini-Mental State Examination. ADAS-cog = AD assessment scale-cognitive subscale. The significance of differences between cognitive state (MMSE, ADAS-cog) values of the groups were calculated by an unpaired t test for C vs. AD (a) and for C vs. AD₁, and by a paired t test for AD₁ vs. AD₂ (b);
*= p < 0.001.

As shown in FIG. 4, Control, AD1 patient and AD2 patient values (mean±S.E.M.), respectively, were 63,621±3056, 63,901±3130 and 163,273±4864 for C4b, 62,958±8,945, 86,911±10,020 and 215,660±23,018 pg/ml for C3d, 82,681±3,921, 84,385±12,808 and 548,930±77,855 pg/ml for complement factor B, 85,716±6181, 85,123±4836 and 247,574±10,740 pg/ml for factor B fragment Bb, 24,347±2350, 22,212±1866 and 70,039±5245 pg/ml for C3b, 400±43.4, 359±31.7 and 832±87.2 pg/ml for C5b-C9 TCC; 1272±61.4, 757±33.6 and 409±15.4 pg/ml for CD59, and 32,123±1733, 13,352±803 and 4369±320 pg/ml for DAF.

The results of longitudinal studies showed no differences between the AD1 preclinical stage and those of matched controls in the mean ADE levels of any of the six complement proteins assessed (see FIG. 4). As for complement proteins, there were no differences between ADE levels of cytokines at the AD1 preclinical stage and those of matched controls. In contrast, mean ADE levels of all complement proteins and cytokines quantified were significantly higher at the AD2 stage of moderate dementia than at the AD1 preclinical stage and for controls. In contrast, ADE levels of membrane-associated complement system regulatory proteins were significantly lower at both AD1 preclinical state and AD2 moderate dementia stage compared to controls (see CD59 and DAF in FIG. 4).

These results showed that ADE levels of C4b, C3d, Factor B, Fragment Bb, C3b, and C5b-C9 TCC are increased in subjects that have been diagnosed with neurological disease but not before diagnosis when cognitively intact. These results further showed that ADE levels of complement regulatory proteins are decreased in subjects prior to being diagnosed with neurological disease and may predict neurological disease. These results demonstrated that the methods of the present invention are useful for detecting biomarkers and measuring biomarker protein levels in astrocyte-derived exosomes. These results further demonstrated that the methods of the present invention may be used to detect exosomal complement biomarkers associated with pathogenesis of neurological diseases, including Alzheimer's disease. These results further showed that methods of the present invention are useful for prognosis, diagnosis, treating or monitoring treatment of exosomal complement abnormalities associated with neurological diseases. The results suggested that the methods of the present invention would be useful for preventing and/or treating neurological diseases.

### Example 3

### Astrocyte-Derived Exosomal Biomarkers are Prognostic for Neurological Disease

A study was performed to detect and measure levels of various biomarkers of the present invention in astrocyte-derived exosomes (ADE) from subjects before and after the development of neurological disease as follows. Participants in four distinct groups were evaluated clinically and by mental status testing at the time of entry into the study and then annually for 36 months: patients with an established diagnosis of mild to moderate Alzheimer's disease (AD, n=20), cognitively normal controls who were age- and gender-matched to the AD group (controls, n=20), subjects with stable mild cognitive impairment (MCI) that did not change during the 36 month study (MCIS, n=20), and subjects who converted within 3 years from MCI to AD dementia (MCIC, n=20) (Table 2). All participants in the MCIS and MCIC groups had been identified through the Alzheimer Disease Cooperative Study (ADCS) Biomarker Core at University of California, San Diego (UCSD). Subjects in the AD and control groups were from the Jewish Home of San Francisco (JHSF). Cognitive normality of controls was based on MMSE, level of activities of daily living and capacity to provide a coherent history and list of medications. Co-morbidities in the four groups were principally hypertension and type 2 diabetes mellitus and were equally distributed in the MCIS and MCIC groups.

Mental status testing was conducted with the Mini-Mental State Examination (MMSE) as described in Goetzl E J et al. Ann Neurol. 2018; 83:544-52. Study patients with MCI or mild to moderate dementia with high probability of AD had a Clinical Dementia Rating global score of 0.5 or 1.0 according to the NIA-Alzheimer's Association and International Working Group-2 criteria (Sperling R A et al. Alzheimers Dement. 2011; 7:280-92). All patients with MCI or AD had an abnormal cerebrospinal fluid (CSF) level of amyloid β-peptide (Aβ) 1-42, that supported their diagnosis (Shaw L M et al. Ann Neurol. 2009; 65:403-13).

All participants with MCI donated blood for exosome analyses at the time of entry into the UCSD study and those with AD and their controls had donated blood over the same period of time at JHSF. Five ml of venous blood were drawn by syringe into 0.5 ml of saline with EDTA, incubated for 10 minutes at room temperature, and centrifuged for 15 minutes at 2,500×g. Plasma samples were stored in 0.25 ml aliquots at −80° C.

### Enrichment of Plasma Astrocyte-Derived Exosomes (ADEs) for Extraction and ELISA Quantification of Proteins

Aliquots of 0.25 ml plasma were incubated with 0.1 ml thromboplastin D (Thermo Fisher Scientific, Inc., Waltham, Mass., USA), followed by addition of 0.15 ml of calcium- and magnesium-free Dulbecco's balanced salt solution (DBS⁻²) with protease inhibitor cocktail (Roche, Indianapolis, Ind., USA) and phosphatase inhibitor cocktail (Thermo Fisher Scientific, Inc.) (DBS⁺⁺), as described [8]. After centrifugation at 3000×g for 30 min at 4° C., total exosomes were precipitated from resultant supernatants with 126 uL per tube of ExoQuick (System Biosciences, Mountain View, Calif., USA) and centrifugation at 1500×g for 30 min at 4° C. To enrich ADEs, total exosomes were resuspended in 0.35 ml of DBS⁻² and incubated for 60 min at room temperature with 1.5 μg of mouse anti-human Glutamine Aspartate Transporter (GLAST) (ACSA-1) biotinylated antibody (Miltenyi Biotec, Inc., Auburn, Calif.) in 50 μL of 3% BSA (1:3.33 dilution of Blocker BSA 10% solution in DBS⁻² [Thermo Fisher Scientific, Inc.]) per tube with mixing, followed by addition of 10 μl of streptavidin-agarose Ultralink resin (Thermo Fisher Scientific, Inc.) in 40 μL of 3% BSA and incubation for 30 min at room temperature with mixing. After centrifugation at 800×g for 10 min at 4° C. and removal of the supernate, each pellet was re-suspended in 100 μL of cold 0.05 M glycine-HCl (pH 3.0) by gentle mixing for 10 sec and centrifuged at 4000×g for 10 min, all at 4° C. Supernatants then were transferred to clean tubes containing 25 μL of 10% BSA and 10 μL of 1 M Tris-HCl (pH=8.0) and mixed before addition of 365 μL of mammalian protein extraction reagent (M-PER) (ThermoFisher Scientific) with protease and phosphatase inhibitors. Resultant 0.5 ml lysates of ADEs were stored at −80° C.

ADE proteins found to be abnormal in AD previously [23] were quantified by ELISA kits for human tetraspanning exosome marker CD81, complement fragment C4b, DAF (CD55) (American Research Products-Cusabio, Waltham, Mass., USA), membrane cofactor protein (CD46), CR1 (American Research Products-Cloud-Clone Corp., Waltham, Mass.), complement fragment C3b, C1q portion of the C1 complement complex (Abcam, Inc., Cambridge, Mass.), Bb fragment of complement factor B (Quidel-Microvue, San Diego, Calif.), complement fragment C5b and terminal complement complex C5b-C9 (Elabscience, Bethesda, Md.), CD59, mannose-binding lectin (MBL) (Ray Biotech, Inc., Norcross, Ga.), and complement factor D (ThermoFisher-Invitrogen, LaFayette, Colo.). The mean value for all determinations of CD81 in each assay group was set at 1.00 and relative values of CD81 for each sample were used to normalize their recovery.

### Statistical Analyses

The Shapiro-Wilks test showed that data in all sets were distributed normally. Statistical significance of differences between means for cross-sectional groups AD and control, MCIS and control, MCIC and MCIS, and MCIC and AD were determined with an unpaired Student's t test, including a Bonferroni correction (Prism 6; GraphPad Software, La Jolla, Calif., USA). ROC analyses were conducted to assess the sensitivity of complement proteins in distinguishing among the subgroups of participants (Prism 6; GraphPad Software).

The mean ADE levels of all complement effector proteins for the MCIC patients who converted to dementia after three years were significantly higher than those for the MCIS patients who remained cognitively stable at the level of MCI after three years (Table 2, FIG. 1). Complement effector proteins with elevated levels in MCIC patients included C1q and C4b of the classical pathway, factor D and fragment Bb of the alternative pathway and C5b, C3b and C5b-C9 terminal complex of both pathways. Levels of four of the ADE complement effector proteins show no overlap between the two groups of MCI subjects. The exception was mannose-binding lectin (MBL) of the lectin complement pathway, where there was no difference between ADE levels of the two MCI groups. Inversely, mean ADE levels of all membrane-localized complement inhibitory proteins for the MCIC patients were significantly lower than those for the MCIS patients (FIG. 2). Significant differences in mean ADE levels of complement effector and inhibitory proteins separate MCIC patients from cognitively normal controls (FIGS. 1 and 2). Similar differences between AD patients and their controls in ADE levels of all effector and regulatory proteins of the complement system, except MBL, were significant.

TABLE 2
Demographic and cognitive characteristics of the study participants

| | Total | Male/ | Age | MMSE, | MMSE, end |
|---|---|---|---|---|---|
| Diagnosis | number | Female | (mean + S.D.) | entry | (3 years) |
| Control | 20 | 12/8 | 70.8 ± 5.34 | 29.2 ± 0.45 | NA |
| MCIS | 20 | 13/7 | 68.7 ± 7.76 | 29.0 ± 0.26 | 28.8 ± 0.33 |
| MCIC | 20 | 11/9 | 75.4 ± 6.82 | 27.4 ± 0.29 | 17.7 ± 0.70* |
| AD | 20 | 12/8 | 71.1 ± 6.90 | 23.9 + 0.75* | NA |

MMSE, mini-mental state examination on entry into the study and at the three-year end of the study, mean ± S.E.M.; MCIS, subjects with mild cognitive impairment that was stable over the three-year study; and MCIC, subjects with mild cognitive impairment on entry into the study that converted to dementia by the end of the three-year study. The significance of differences in MMSE between the MCIS and MCIC groups and between the Control and AD groups was calculated with an unpaired t test;
*p < 0.001.
NA = not applicable to the Control and AD groups where cognitive evaluation was conducted once on entry into the study.

There were nearly universally significant differences in ADE complement protein levels between MCIC and MCIS participants and between AD patients and controls (FIGS. 1 and 2, Table 3). Although ADE levels of seven complement proteins reliably distinguished MCIS participants from controls, ADE levels of only C4b, Bb and CD59 significantly distinguished patients with AD from those with MCIC, who are at an early stage of AD and converted to AD within three years. The complement proteins that most consistently distinguished among groups were Bb, C3b, C4b, DAF and CD46 (Table 3). C5b, factor D and CR1 all failed to distinguish MCIS participants from controls or AD patients from subjects with MCI converting to dementia (MCIC) (see Table 3).

TABLE 3
Receiver operating characteristic (ROC) evaluation of complement
protein sensitivity in distinguishing clinical subgroups

| Complement | Sensitivity, % (mean ± S.E.M.) | | | |
|---|---|---|---|---|
| protein | MCIS vs. Control | MCIC vs. MCIS | AD vs. Control | AD vs. MCIC |
| Bb | 97.8 ± 0.018 | 100 | 93.0 ± 0.038 | 85.5 ± 0.059 |
| C3b | 96.3 ± 0.028 | 100 | 98.0 ± 0.017 | 65.8 ± 0.088 |
| C1q | 78.3 ± 0.075 | 100 | 99.3 ± 0.009 | — |
| C4b | 66.3 ± 0.087 | 99.8 ± 0.004 | 99.0 ± 0.011 | 100 |
| C5b | — | 89.3 ± 0.052 | 93.8 ± 0.036 | — |
| TCC | 72.6 ± 0.082 | 92.8 ± 0.039 | 95.0 ± 0.031 | — |
| Factor D | — | 94.3 ± 0.033 | 95.3 ± 0.029 | — |
| DAF | 93.4 ± 0.036 | 93.0 ± 0.045 | 100 | 65.9 ± 0.092 |
| CD46 | 92.0 ± 0.052 | 94.3 ± 0.049 | 85.3 ± 0.061 | 66.3 ± 0.089 |
| CD59 | 77.5 ± 0.079 | 69.0 ± 0.084 | 100 | 75.5 ± 0.075 |
| CR1 | — | 79.3 ± 0.072 | 83.0 ± 0.064 | — |

All values of sensitivity less than 60% are represented by dashes. All values for MBL were lower than 60%.

These results showed that increases in ADE levels of C1q, C4b, factor D, fragment Bb, C5b, C3b, and C5b-C9 are diagnostic for subjects with MGIC. These results further showed that ADE levels of C4b, Bb and CD59 are useful for distinguising subjects with AD from those with MGIC, who are at an early stage of AD and converted to AD within three years. These results further showed that ADE levels of complement regulatory proteins are decreased in subjects prior to being diagnosed with neurological disease and may predict neurological disease. These results demonstrated that the methods of the present invention are useful for detecting biomarkers and measuring biomarker protein levels in astrocyte-derived exosomes. These results further demonstrated that the methods of the present invention may be used to detect exosomal complement biomarkers associated with pathogenesis of neurological diseases, including Alzheimer's disease, mild cognitive impairment (MCI), and conversion of mild cognitive impairment to dementia or Alzheimer's disease. These results further showed that methods of the present invention are useful for prognosis, diagnosis, treating or monitoring treatment of exosomal complement abnormalities associated with neurological diseases. The results suggested that the methods of the present invention would be useful for preventing and/or treating neurological diseases.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method comprising: a) providing a biological sample comprising astrocyte-derived exosomes from a subject; b) enriching the sample for astrocyte-derived exosomes; and c) detecting the presence of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) in the sample.

2. The methods of claim 1, wherein the biological sample is selected from the list consisting of whole blood, plasma, serum, lymph, amniotic fluid, urine, saliva, and umbilical cord blood.

3. The method of claim 1, wherein the marker is a full-size marker or a fragment of the full-size marker.

4. The method of claim 1, wherein the detecting the presence of the marker in the biological sample comprises detecting the amount of the marker in the biological sample.

5. The method of claim 1, further comprising the step of deterrninmg a treatment course of action based on the detection of the one or more biomarkers.

6. The method of claim 1, where in the neurological disease is selected from the group consisting of Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

7. A method comprising: a) providing a biological sample comprising astrocyte-derived exosomes from a subject having a neurological disease or suspected of having a neurological disease; b) isolating astrocyte-derived exosomes from the biological sample; and c) detecting the presence of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) in the exosomes.

8. The method of claim 7, wherein the isolating astrocyte-derived exosomes from the biological sample comprises: contacting the biological sample with an agent under conditions wherein an astrocyte-derived exosome present in the biological sample binds to the agent to form an astrocyte-derived exosome-agent complex; and isolating the astrocyte-derived exosome from the astrocyte-derived exosome-agent complex to obtain a sample containing the astrocyte-derived exosome, wherein the purity of the astrocyte-derived exosomes present in said sample is greater than the purity of the astrocyte-derived exosomes present in said biological sample.

9. The method of claim 7, wherein the agent is an antibody.

10. The method of claim 9, wherein the antibody is an anti-Glutamine Aspartate Transporter antibody.

11. The methods of claim 7, wherein the biological sample is selected from the list consisting of whole blood, plasma, serum, lymph, amniotic fluid, urine, saliva, and umbilical cord blood.

12. The method of claim 7, wherein the marker is a hall size marker or a fragment of the full-size marker.

13. The method of claim 7, wherein the detecting the presence of the marker in the biological sample comprises detecting the amount of the marker in the biological sample.

14. The method of claim 7, further comprising the step of detennining a treatment course of action based on the detection of the one or more biomarkers.

15. The method of claim 7, where in the neurological disease is selected from the group consisting of Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

16. A method for treating a subject having a neurological disease, comprising the steps of: providing a biological sample from a subject suspected of having a neurological disease, wherein the sample comprises astrocyte-derived exosomes; measuring the level of one or more biomarkers selected from the group consisting of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) from the biological sample, wherein an altered level of the one or more biomarkers in the sample relative to the level in a control sample is indicative of a need for treatment; and administering an effective amount of an agent to the subject thereby treating the neurological disease in the subject.

17. The method of claim 16, where in the neurological disease is selected from the group consisting of Alzheimer's disease (AD), vascular disease dementia, mild cognitive impairment (MCI), frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

18. The method of claim 16, wherein the agent is a recombinant complement control protein selected from the group consisting of recombinant membrane inhibitor of reactive lysis (CD59), recombinant membrane cofactor protein (CD46), recombinant decay-accelerating factor (DAF) and recombinant complement receptor type 1 (CR1).

19. The method of claim 16, wherein the agent is neutralizing monoclonal antibodies to effector complement components or their receptors, decoy complement receptors or receptor antagonists, or an esterase inhibitor.
